(19) United States
(12) Patent Application Publication
(10) Pub. No.: US 2007/0041864 A1
(43) Pub. Date: Feb. 22, 2007

(54) Methods and apparatus for hot air sterilization of medical instruments

(76) Inventors: Michael J. Forsyth, Stow, OH (US); Gregory L. Smith, Cuyahoga Falls, OH (US)

Correspondence Address: KATTEN MUCHIN ROSENMAN LLP, 575 MADISON AVENUE, NEW YORK, NY, 10022-2585, US

(21) Appl. No.: 11/501,992

(22) Filed: Aug. 10, 2006

Related U.S. Application Data
(60) Provisional application No. 60/707,432, filed on Aug. 10, 2005.

(51) Int. Cl. A61L 2/24; A61L 2/04

(52) U.S. Cl. 422/1; 422/62; 422/307; 422/3

(57) ABSTRACT

A heat sterilizer sterilizes a load held in a sterilizing chamber at a predefined temperature over a shortened sterilization period by using predefined heating and cooling profiles. The sterilizer includes a temperature sensor coupled to a controller for sensing the temperature within the sterilizing chamber, where the controller is further adapted to monitor an output of the temperature sensor and maintain the temperature within the sterilizing chamber according to the predefined heating profile, the predefined temperature, or the predefined cooling profile, and where the controller is further adapted to specify an error condition if the controller fails to maintain the temperature within the sterilizing chamber according to the predefined heating profile, the predefined temperature, or the predefined cooling profile.

### BACKGROUND OF THE INVENTION

1. Field of the Invention

The invention relates to hot air sterilization and, in particular, to self-contained hot air sterilizers with forced cooling to reduce sterilization cycle times.

2. Description of the Prior Art

Certain medical practices such as small dental and/or orthodontic practices require the availability of large numbers of instruments during a day in the treatment of many patients. In order to reduce the total number of instruments needed to support such practices, sterilization turn around times must be as short as possible. Accordingly, sterilizers with rapid cooling cycles are valuable to such individual practices.

Dry heat sterilization is increasingly becoming the method of choice for sterilizing medical instruments with carbide and carbon steel components. Such sterilization provides safety for all instruments which are chemically and moisture sensitive by introducing no foreign substances and by effectively eliminating moisture in the sterilizing atmosphere.

Medical dry heat sterilizers previously available for use in a practitioner's office typically have used the method of conductive heat transfer both to elevate the instrument loads to a sterilizing temperature and thereafter reduce the temperature of the instruments to a usable level.

Other approaches have been suggested. One suggested approach has been to provide forced air heating for sterilization but to make no provision for cooling. Another approach has been to provide forced air heating within a sterilizing chamber followed by forced air cooling of the outside of the sterilizing chamber walls. Still another suggested approach is the forced air heating and cooling of the contents of a sterilizing chamber by a single fan along a single air flow path through the sterilizing chamber. One disadvantage suffered by all of these approaches with forced air cooling cycles is that cooling air is circulated through the sterilizer in such a way that it cools the sterilizing air heat source as well as the instruments and other objects being sterilized. In some approaches, cooling air is directed across the heat source before it is passed into the sterilizing chamber and therefore cools the heat source before cooling the contents of the chamber. In some approaches the cooling air must also cool the fan which had been used to circulate heated air through the sterilizing chamber. Cooling time for such sterilizers is extended by the time needed to additionally cool the heat source, and/or the hot air circulating fan.

### SUMMARY OF THE INVENTION

The present invention provides an improved dry air sterilization system and methods for sterilizing, for example, dental or surgical tools. Not including heat-up and cool down time, the system is adapted to sterilize in a short (e.g., three minute) cycle. The system may use a temperature monitored forced air cool down process. Filtered air may be used during cool down through the use of a replaceable HEPA filter that complies with ANSI/AAMI ST50: 2004. A safety door lock feature may be employed to lock the door when the unit reaches a predefined threshold temperature (e.g., 50° C.) and keep it locked until the unit cools down (e.g., to 45° C.). The system may include a communications port to allow an operator connection to a personal computer (PC) or serial printer to download cycle data logs. Software may be used to monitor all cycle parameters and provide diagnostic error codes as well as to trigger an audible alarm when appropriate, e.g., when a process error has been detected. Many operational parameters may be configured by the operator.

Other features and aspects of the present invention will become more fully apparent from the following detailed description of exemplary embodiments, the appended claims, and the accompanying drawings.

### BRIEF DESCRIPTION OF THE DRAWINGS

FIGS. 1 to 18 depict an example embodiment of the present invention in operation.

FIG. 19 is a time versus temperature graph depicting an example heating/cooling cycle profile according to some embodiments of the present invention.

FIG. 20 depicts a view of a door safety interlock in a disengaged position according to some embodiments of the present invention.

FIG. 21 depicts a view of a door safety lock in an engaged position according to some embodiments of the present invention.

FIG. 22 depicts a view of the location of the air filter and operator control/status panel according to some embodiments of the present invention.

FIG. 23 depicts a close-up view of the operator control/status panel according to some embodiments of the present invention.

FIG. 24 depicts a view of a sterilization chamber and operator control/status panel according to some embodiments of the present invention.

FIG. 25 depicts a view of an empty sterilization chamber according to some embodiments of the present invention.

FIG. 26 depicts a view of a loaded sterilization chamber according to some embodiments of the present invention.

FIG. 27 depicts a representation of air circulation in an empty sterilization chamber according to some embodiments of the present invention.

FIG. 28 depicts a view of a loaded sterilization chamber and the location of a control/display temperature sensor (RTD) according to some embodiments of the present invention.

FIG. 29 depicts a view of a lower portion of a sterilization chamber with a lower panel removed according to some embodiments of the present invention.

FIG. 30 depicts a schematic diagram of a temperature sensor suitable for use with some embodiments of the present invention.

FIG. 31 depicts a close-up view of a COM port suitable for use with some embodiments of the present invention.

FIG. 32 depicts a top View of an enclosure suitable for use with some embodiments of the present invention.

FIG. 33 depicts a close-up view of a sterilization chamber door handle suitable for use with some embodiments of the present invention.

FIG. 34 depicts a left side view of an enclosure suitable for use with some embodiments of the present invention.

FIG. 35 depicts a front view of an enclosure suitable for use with some embodiments of the present invention.

FIG. 36 depicts a right side view of an enclosure suitable for use with some embodiments of the present invention.

FIG. 37 depicts a rear view of an enclosure suitable for use with some embodiments of the present invention.

FIG. 38 depicts a close-up view of a sterilization chamber identification information suitable for use with some embodiments of the present invention.

FIG. 39 is a block diagram depicting an example software system architecture according to some embodiments of the present invention.

FIG. 40 is a block diagram depicting an example hardware system architecture according to some embodiments of the present invention.

FIG. 41 is a block diagram depicting an example state machine for a system start-up process according to some embodiments of the present invention.

FIG. 42 is a block diagram depicting an example state machine for a user/service menu process according to some embodiments of the present invention.

FIG. 43 is a block diagram depicting an example state machine for a sterilization sequence according to some embodiments of the present invention.

FIG. 44 is a screenshot of an information window displaying the meanings of various combinations of colored system status LEDs according to some embodiments of the present invention.

### DETAILED DESCRIPTION OF THE PREFERRED EMBODIMENTS

While the present invention is illustrated and described in a preferred embodiment, the device may be produced in many different configurations, forms and materials. There is depicted in the drawings, and will herein be described in detail, a preferred embodiment of the invention, with the understanding that the present disclosure is to be considered as an exemplification of the principles of the invention and the associated functional specifications for its construction is not intended to limit the invention to the embodiment illustrated. Those skilled in the art will envision many other possible variations within the scope of the present invention.

Reference will now be made in detail to embodiments of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity only, directional terms, such as upper, lower, top, bottom, left, right, up, down, over, above, below, beneath, rear, and front may be used with respect to the drawings. These and similar directional terms should not be construed to limit the scope of the invention in any manner. The words “connect,” “couple,” and similar terms with their inflectional morphemes do not necessarily denote direct and immediate connections, but also include connections through mediate elements or devices. Furthermore, such terms as “unit,” “system,” “device,” “chamber,” “sterilization system,” “sterilizer,” and the like, are used interchangeably. The following detailed description is of the best mode or modes of the invention presently contemplated. As indicated above, such description is not intended to be understood in a limiting sense, but to be an example of the invention presented solely for illustration thereof, and by reference to which in connection with the following description and the accompanying drawings one skilled in the art may be advised of the advantages and construction of the invention.

Embodiments of the system of the present invention may include, for example, Class II, tabletop, (forced air) convection type, software controlled, batch process dry heat sterilizers for medical instruments or other load types. Such sterilizer systems may be supplied with plier racks for convenient placement and positioning of the load (e.g., instruments) to be sterilized. Such systems provide the operator with safe and effective means of sterilizing unbagged loads that can withstand typical dry heat sterilization. These systems are easily operated and may be implemented so as to not require any user interaction. Convenient rack systems may be provided to simplify loading and unloading.

As indicated above, the present invention is described herein with reference to a specific implementation for illustrative purposes. Thus, it is important to note that the particular features and specific embodiments described herein are merely exemplary and that any practicable subset of such features, whether explicitly described as a subset or not, may comprise the present invention. The present invention provides an improved dry air sterilization system and methods for sterilizing, for example, medical instruments. Not including heat-up and cool down time, the system is adapted to sterilize in a short (e.g., three minute) cycle. The system may use a temperature monitored, forced air cool down process. Filtered air may be used during cool down through the use of a replaceable HEPA filter that complies with ANSI/AAMI ST50: 2004. A safety door lock feature may be used to lock the door when the unit reaches a predefined threshold (e.g., 50° C.) and may not unlock until the unit cools down (e.g., to 45° C.). The system may include a communications (COM) port to allow an operator connection to a PC or serial printer to download cycle data logs. Software may be used to monitor all cycle parameters and provide diagnostic error codes as well as to trigger an audible alarm when appropriate, e.g., when a process error has been detected. Many operational parameters may be configured by the operator.

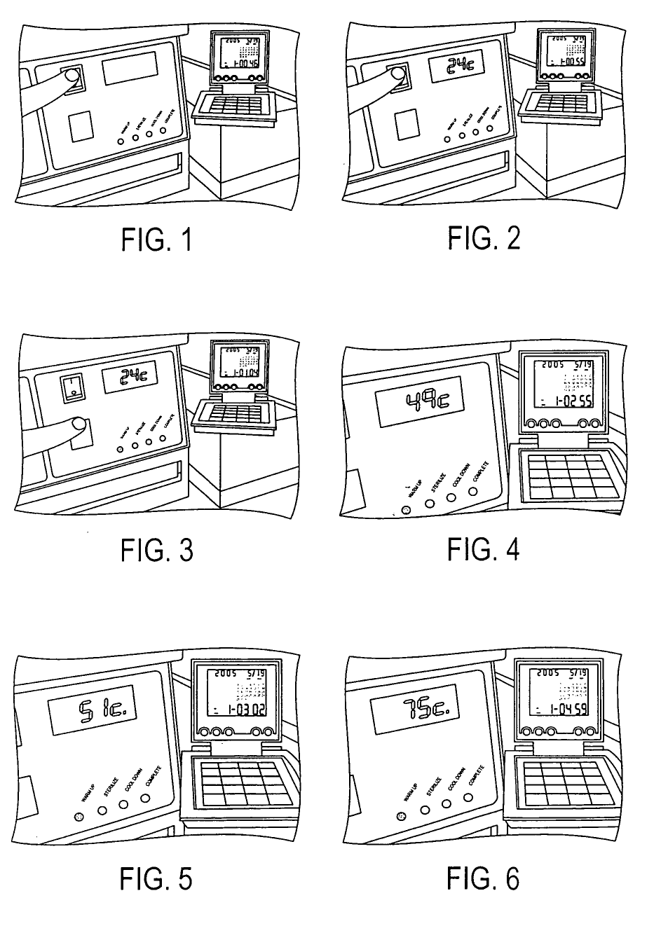

Turning to FIG. 1, to activate the device, the “standby/on” switch is toggled into the “on” position. This activates the LED display panel (as shown in FIG. 2), the cycle start switch and the communications port (if a printer or PC is connected). A PC is connected to the COM port in the example shown in the figures. The display will numerically indicate the current temperature in the chamber in degrees Celsius or Fahrenheit based on an operator configured setting. In the particular example embodiment depicted in FIGS. 1 and 2, no indicator lights are illuminated at this point.

Depressing and releasing the spring loaded, momentary “cycle start” switch will initiate the software-controlled cycle as depicted in FIG. 3. Once the cycle start switch is pressed, the yellow LED labeled “warm up” illuminates also as shown in FIG. 3. The heater and the blower are activated. The blower draws air from the chamber through slots located below each of the four designated plier rack channels past a mounted RTD sensor, which monitors the air temperature as it is being drawn from the chamber. This temperature (e.g., 49° C. in FIG. 4) is displayed on the red LED display.

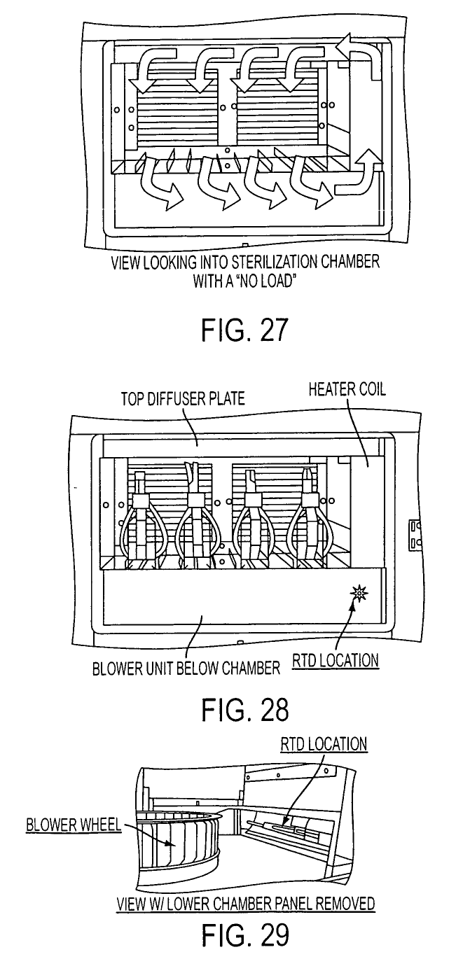

The air is forced through the resistance-type coil heater located to the right of the chamber, elevating the air temperature prior to re-entering the sterilization chamber. The heated air is forced back into the sterilization chamber through a diffuser plate located on the topside of the chamber. The heated air continuously flows around the load as shown in FIG. 27.

Referring to FIG. 5, note that once the chamber exceeds 50° C. in this example embodiment, a dot appears in the lower right of the display. The dot on the display indicates that the safety interlock switch has been engaged. The safety interlock switch is an option that provides a mechanical lock, which engages when the chamber temperature warms up past 50° C. and disengages when the chamber cools below 45° C.

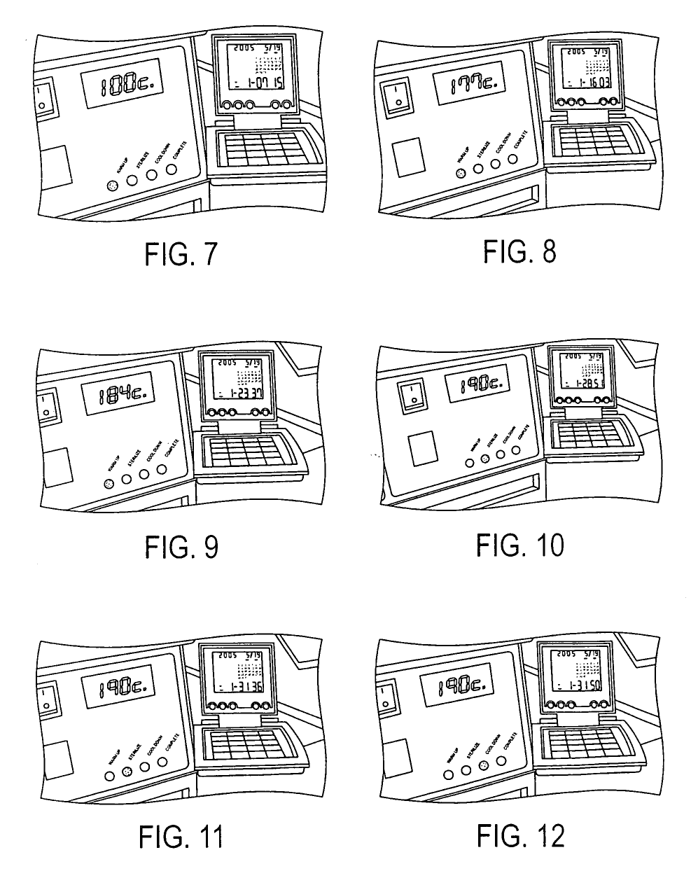

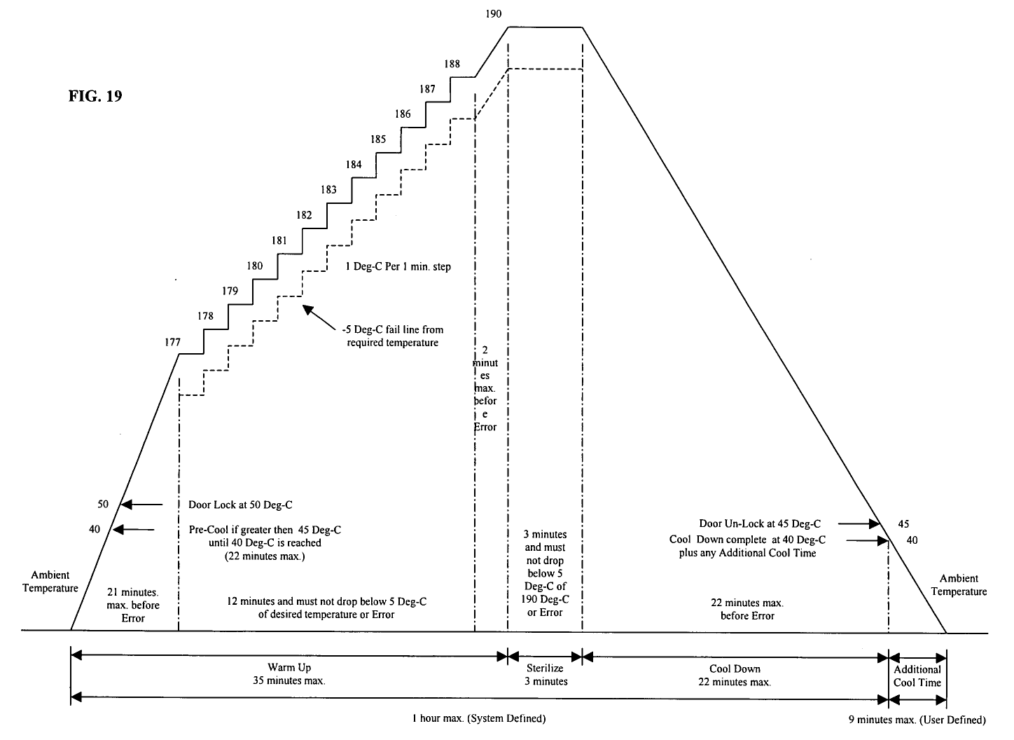

The chamber temperature continues to increase during the warm up (heat up) phase as shown in the progression from FIG. 6 to FIG. 7 to FIG. 8 to FIG. 9. At 177° C. (FIG. 8), the heating is controlled according to a predefined time and temperature profile depicted in the cycle profile graph of FIG. 19.

As shown in FIG. 10, once the RTD sensor detects a temperature of 190° C. (374° F.), the yellow LED is extinguished (e.g., the warm-up phase has completed) and the amber LED labeled “sterilize” is illuminated. During this phase, the blower continues running and re-circulating the heated air. The heater coil is programmed to shut off when a temperature of 190° C. (374° F.) is measured at the RTD. The heater coil is re-activated when the RTD detects a temperature of 188° C. (370° F.). The activation and de-activation of the heater, based on RTD readings, is continued for the three (3) minute sterilization phase as shown in FIG. 11 (e.g., note the timer on the display of the PC connected to the chamber relative to the same timer in FIG. 10).

Temperature studies have shown, that by controlling the heater with the RTD limits of 188° C. to 190° C., the chamber air temperature will not fall below 190° C. (374° F.). If the RTD temperature ever falls below 185° C. (365° F.), the cycle will fail.

At the end of this 3-minute period, the amber LED will turn off (e.g., indicating the completion of the sterilization phase) and the blue LED labeled “cool down” will illuminate, indicating that the “Cool Down” phase has begun as shown in FIG. 12. At this time, the heater circuit is disabled. Ambient room air is drawn in to the chamber by a 5-bladed AC fan located at the rear left side of the unit. Air is drawn through a replaceable HEPA filter (filtration efficiency of 99.97% for 0.3 micron particles) located on the bottom of the unit. The cool, filtered air is transported from the filter to the chamber by way of a sealed duct running up the rear of the unit.

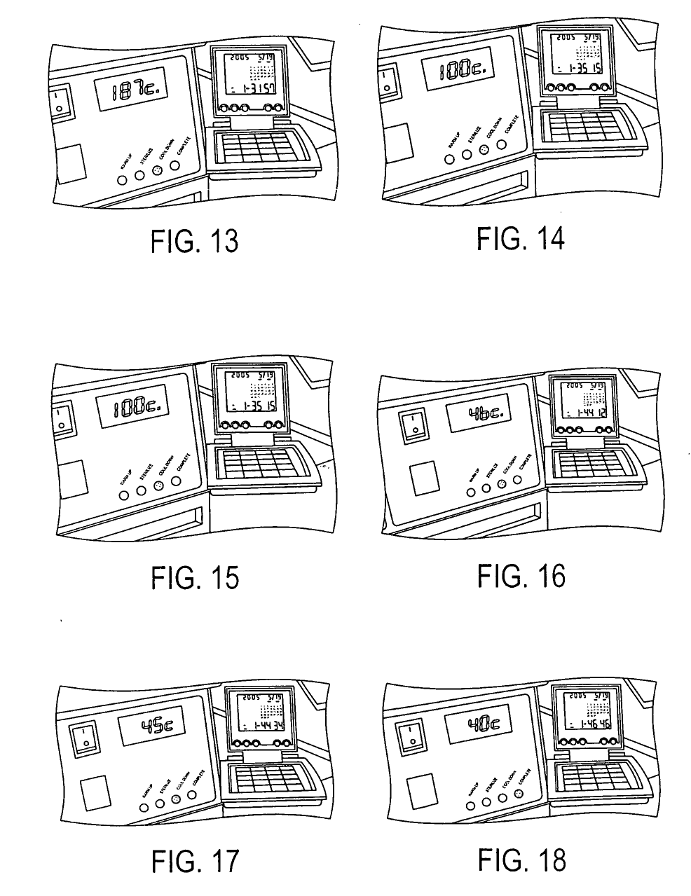

Cool filtered air is continuously re-circulated into the chamber to cool the load. The chamber temperature is continually monitored by the RTD sensor and displayed on the LED display panel as shown in the sequence of FIGS. 12 to 16. As the cool, filtered air is introduced into the chamber, the hot air is exhausted through the louvered exhaust port located on the right, rear side. The exhausted air is vented outside of the unit into the room. Notice that the small dot remains visible on the lower right corner of the display.

Once the chamber cools to 45° C., the safety interlock solenoid is disengaged. The dot goes away on the lower right side of the display as shown in FIG. 17. The cool down phase is complete once the RTD detects chamber air temperatures of 40° C. (104° F.) bringing the load to a safe handling temperature. As shown in FIG. 18, the processor turns the blower motor assembly, the blue LED, and the cool-down fan “off” and illuminates the green LED labeled “complete” indicating the successful completion of the sterilization cycle. An audible tone will signal the operator that the sterilization has been completed and that it is now safe to open the door. To open the door, the handle is turned clockwise. Once the door is opened, the green LED turns off. If a print out of the cycle (in terms of time v. temperature) is desired, one can be printed at this point. A print out of the last cycle can be printed anytime prior to starting a new cycle. In some embodiments, any number of cycles may be stored for later review.

Turning to FIG. 18, a graphical representation of the cycle shows the dynamics during warm up (heat up), sterilize (exposure), and cool down.

In case the system malfunctions or the cycle is disrupted, an error code is displayed in the display window. The system is adapted to provide diagnostic error codes based on time, temperature and switch monitoring. Example error code definitions and corrective action steps for each of the error codes are described in detail below.

The system may incorporates a number of features to enhance the safe operation by the operator and to comply with safety standards. These features include a safety door power cut-off switch, time out limiters, a safety temperature limit switch, replaceable inline fuses, and a safety door interlock.

The safety door power cut-off switch is a momentary switch located beneath the door on top of the control panel. If the door is opened during any phase in the cycle, an error code and audible tone will result and the cycle will terminate.

The time out limiters are predefined timers that prevent the system from operating indefinitely. For example, if the “warm up” cycle exceeds 34 minutes or the “cool down” cycle exceeds 23 minutes, an error code will display and the cycle will terminate. An error during warm up could be an indication of improper loading of instruments, objects blocking air flow slots in chamber or a heater assembly failure. An error during cool down could indicate a malfunction of the cooling fan, the blower assembly is in need of maintenance, or the HEPA filter requires replacement because the airflow through the filter may be restricted.

A safety, temperature limit switch, located above the heater, will shut down the sterilizer if the air temperature reaches above 371° C. or 700° F. This feature will prevent the chamber from overheating in the event of a blower or RTD sensor malfunction, or airflow restriction.

115 VAC and 230 VAC units contain replaceable inline fuses.

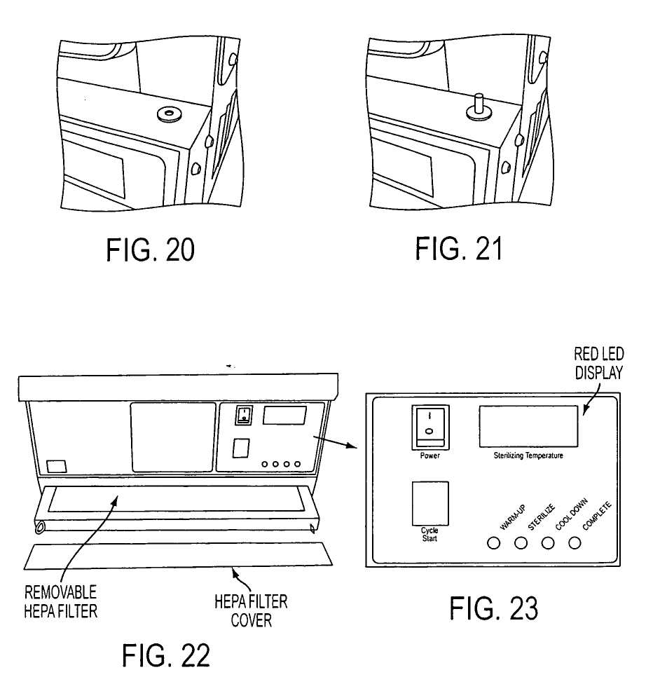

The safety door interlock includes a solenoid operated shot pin that activates when the chamber temperature reaches a predefined threshold (e.g., 50° C./122° F.) during the warm-up cycle. The interlock does not release the door until the chamber reaches a safe air temperature (e.g., 45° C./113° F.) The solenoid and shot pin are located in the right side edge of the control panel as shown in FIGS. 20 and 21. The shot pin extends into the bottom right corner of the door, thus preventing access to the chamber until the solenoid retracts the pin. FIG. 20 depicts the safety door interlock in an open/unlocked position. FIG. 21 depicts the safety door interlock in a closed/locked position.

The particular example embodiment depicted in the figures has the following physical characteristics. The width is 18¾″ (47.6 cm), the depth is 20″ (50.8 cm), the height is 22¾″ (57.8 cm), the weight is 90 LBS.(40.8 Kg), the color is off white, the construction is of steel. The chamber dimensions include a width of 12½″ (31.8 cm), a depth of 9″ (22.9 cm), and a height of 6½″ (16.5 cm). The construction is of stainless steel. The power consumption is 115 Volts, 15 Amps or 230 Volts, 8 Amps. The system uses a grounded outlet. These characteristics are merely exemplary. Many other systems having different physical characteristics may be used to practice the present invention.

FIGS. 22 through 38 depict different physical attributes of an example embodiment of the system. FIG. 22 depicts the locations of the air filter and the operator control/status panel. The removable filter and filter cover are identified. As indicated above, HEPA filters may be used to ensure that cooling air will not contaminate the sterilized load. FIG. 23 is a close-up view of the operator control/status panel. The LED display that is adapted to show both temperature and error/status codes is identified.

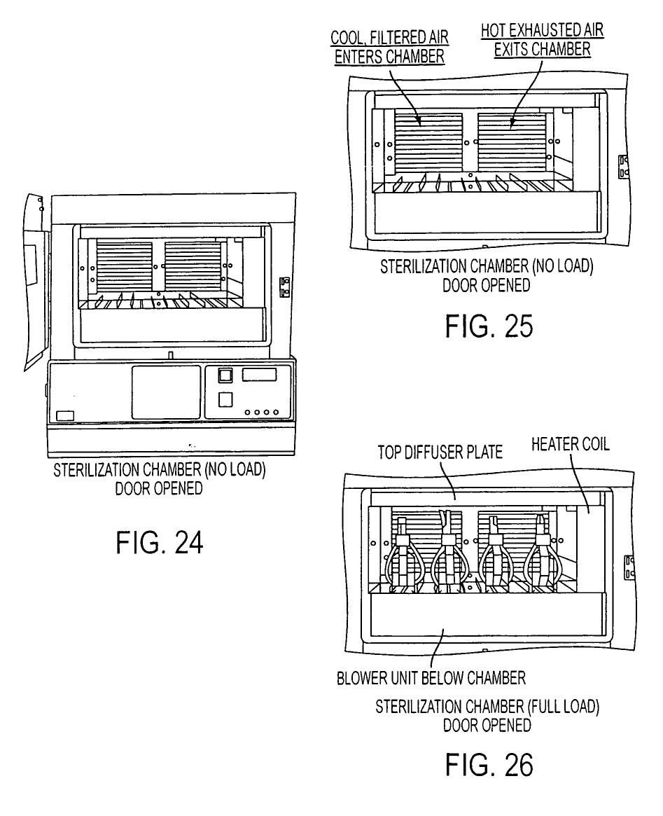

FIGS. 24 to 26 depict views of an open sterilization chamber and operator control/status panel according to some embodiments of the present invention. FIG. 25 depicts an empty sterilization chamber. The vents for cool air entry and hot air exhaust can be seen at the back of the chamber. FIG. 26 depicts a loaded sterilization chamber. The location of the blower unit, heater coil, and top diffuser plate are identified.

FIG. 27 depicts a representation of air circulation in an empty sterilization chamber during heating and sterilization. The arrows display air flow pushed from the blower unit going up through the heater coil on the right side, out into the chamber via the top diffuser plate (e.g., across the space that a load would occupy), and pulled down back to the blower.

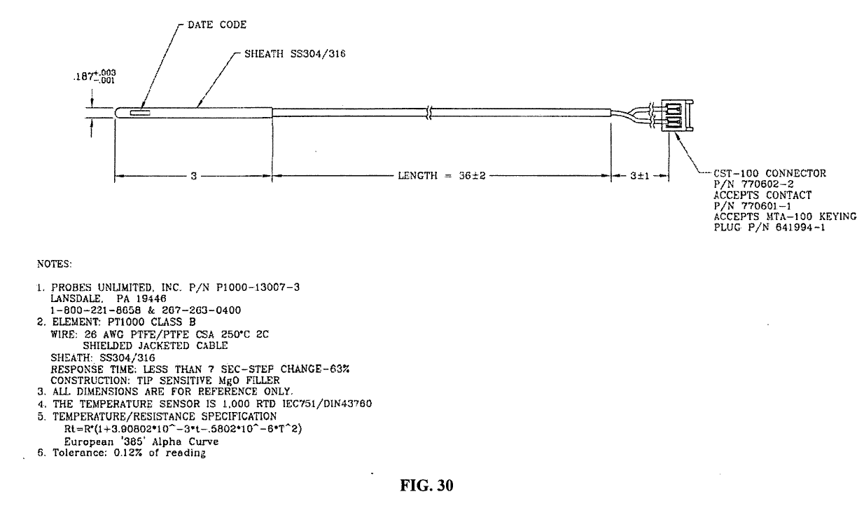

FIG. 28 depicts a view of a loaded sterilization chamber. As shown, the example chamber can hold four plier racks with nine pliers each for a total of 36 pliers. Note that the load is arranged to allow air flow from the diffuser to the blower. FIG. 28 also depicts the location of a control/display temperature sensor (RTD) according to some embodiments of the present invention. Note that the sensor is disposed within the blower unit duct before the heater coil (e.g., upstream from the heater coil) such that air from the chamber flows directly past it. This mounting location ensures that the air temperature is accurately measured and any hot or cold spots within the chamber are avoided. FIG. 29 depicts a close-up of the lower portion of the chamber with the lower panel removed. The blower and the RTD temperature sensor can be seen within the exposed area. The temperature sensor is 1,000 RTD (IEC751/DIN43760). To meet ANSI/AAMI ST50: 2004 qualifications under section 4.6.1.3, the temperature/resistance specification for a suitable sensor is Rt=R° (1+3.90802*10ˆ−3*t−0.5802*10ˆ−6*Tˆ2) with a European ‘385’ alpha curve and a tolerance of 0.12% of the reading. FIG. 30 depicts a schematic diagram of a temperature sensor suitable for use with some embodiments of the present invention.

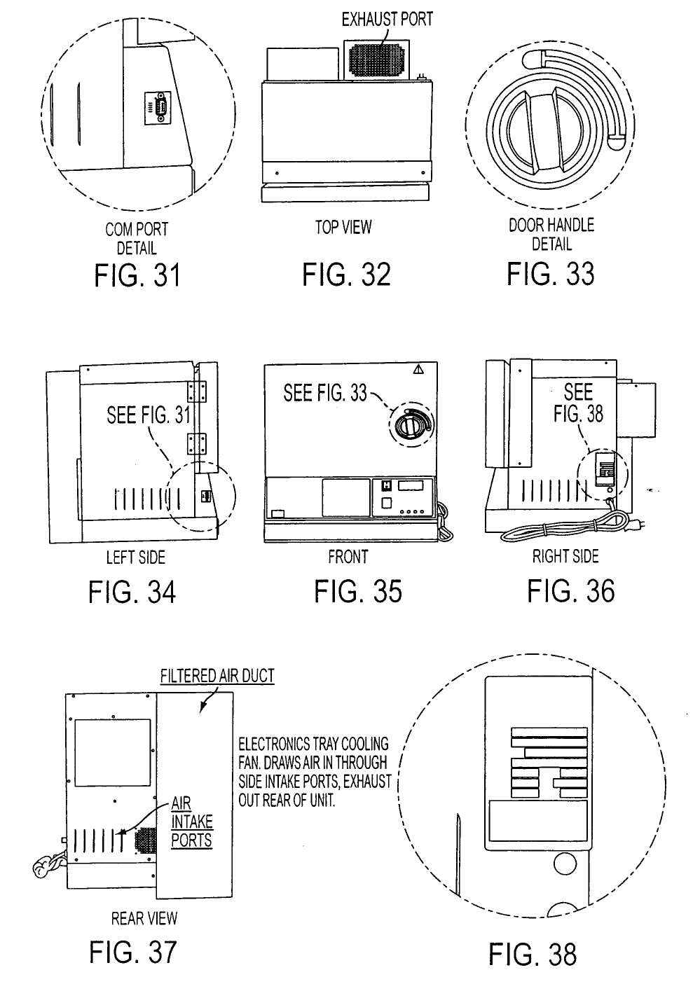

FIGS. 31 to 38 depict various views of the exterior of an enclosure that may be used to house the sterilization chamber and other components. FIG. 31 depicts a close-up view of a detail of FIG. 34. The COM port depicted is suitable for use with embodiments of the present invention. FIG. 32 depicts a top view of the enclosure. Note that the exhaust port or vent is identified. FIG. 33 depicts a close-up view of a detail of FIG. 35. The example sterilization chamber's door handle is depicted. FIGS. 34, 35, 36, and 37 depict left side, front, right side, and rear views, respectively, of the example enclosure. Note that the filtered cool air duct and air intake ports are identified in FIG. 37. Finally, FIG. 38 depicts a close-up view of a portion of the right side (FIG. 36) of the sterilization chamber. An identification information panel is depicted.

Referring back to FIG. 19, operation of the control system is now described. In Step 1, the unit is powered. The system may be plugged into an appropriate power rated receptacle. Note that the unit is always on, but in standby mode while Standby/On switch is set to “Standby.” In Step 2, the inputs are set. This step may include loading tools to sterilize into the chamber, setting the Standby/On switch to “On”, closeing and locking the door, and toggling the “Cycle Start” switch, Once the Cycle Start switch is toggled, switching to Standby or opening the door will cause an error and an error message will be displayed. Note that the sterilizer will not operate while the door is open.

In Step 3, the sterilization process is executed. In Phase 0 of Step 3, a pre-cool down process make be executed. If the internal temperature of the system is above a predefined threshold (e.g., 45 degrees C.), the system's controller will attempt to cool the unit down (e.g., to 40 degrees C.) within a brief (e.g., 22-minute) period. If the temperature is not reduced (e.g., to 40 degrees C.) within the time allowed, an error will be generated and displayed. At the time of Pre-Cool Down, if the temperature is above a second threshold (e.g., 50 degrees C.) and if the door lock is enabled, then the door lock pin will engage. The door pin will disengage at the first threshold (e.g., 45 degrees C.). Note that if the sterilizer is below the first threshold (e.g., <45 degrees C.), then Phase 0 will be skipped. In some embodiments, a maximum cool down time may be defined (e.g., the cool down must complete within 22 minutes otherwise an error is displayed and the process is aborted).

In Phase 1 of Step 3, a warm-up process is executed. This phase may be divided into 3 routines. The three routines may include ramp, stepped and ramp gain. During the first ramp routine, the temperature must reach a third predefine threshold (e.g., 177 degrees C.) within a predefined time period (e.g., 21 minutes) or an error will be generated and displayed. Once the temperature exceeds the second threshold (e.g., 50 degrees C.) and if the door lock is enabled, then the door lock pin will engage. The heat remains on until the third threshold (e.g., 177 degrees C.) is reached. If the unit reaches the third threshold (e.g., 177 degrees C.) within the allocated period, the stepped routine begins. During the stepped routine, the temperature is increased lc per minute from the third threshold (e.g., 177 degrees C.) to a fourth threshold (e.g., 188 degrees C.). During this stepped routine, the temperature is not permitted to drop below a predefined window amount (e.g., 5 degrees C.) of the desired temperature. If the temperature does drop below the desired window temperature or does not reach the fourth threshold within the time allowed, an error will be generated and displayed. During this stepped routine, the heater is switched on when the measured temperature falls below a desired temperature by a second window amount (e.g., 2 degrees C.) and off at the desired temperature. If the unit reaches the fourth threshold (e.g., 188 degrees C.) within the allocated period, the second ramp routine begins. During the second ramp routine, the temperature must reach a fifth threshold (e.g., 190 degrees C.) from the fourth threshold (e.g., 188 degrees C.) within a predefined time period (e.g., 2 minutes), or an error will be generated and displayed. During the second ramp routine, the heater is switched on at the fourth threshold (e.g., 188 degrees C.) and off at the fifth threshold (e.g., 190 degrees C.). A maximum warming time may be predefined (e.g., Phase 1 must complete within 35 minutes otherwise an error is displayed and the process is aborted).

In Phase 2 of Step 3, a sterilization process is executed. During sterilization, the desired temperature (e.g., 190 degrees C.) is maintained for a predefine time period (e.g., 3 minutes). If the temperature drops below a predefined window (e.g., 5 degrees C.) of the desired temperature during sterilization, an error will be generated and displayed. The heater is switched on at the fourth threshold (e.g., 188 degrees C.) and off at the fifth threshold (e.g., 190 degrees C.). During sterilization, if the temperature exceeds the second threshold (e.g., 50 degrees C.) and if the door lock is enabled, then the door lock pin will engage. The door pin will disengage at the first threshold (e.g., 45 degrees C.). A maximum sterilization time may be predefined (e.g., Phase 2 runs for 3 minutes).

In Phase 3 of Step 3, a cool down process is executed. If the internal temperature of the system is above a predefined threshold (e.g., 40 degrees C.), the system's controller will attempt to cool the unit down (e.g., to 40 degrees C.) within a brief (e.g., 22-minute) period plus, in some embodiments, an Additional Cooling Time “ACT” period. If the temperature is not reduced to the predefined threshold within the time allowed, an error will be generated and displayed. If an operator deems the tools need additional cooling, the operator can add additional cooling time (ACT) (e.g., 9 more minutes of cooling in 1-minute increments). This additional cooling is added to the end of the normal cool down cycle and postpones the completion of the cool down state. if the temperature exceeds the second threshold (e.g., 50 degrees C.) and if the door lock is enabled, then the door lock pin will engage. The door pin will disengage at the first threshold (e.g., 45 degrees C.). A maximum cool down time may be predefined (e.g., Phase 3 runs for 22 minutes plus the ACT, for example, a maximum of 9 minutes additional).

In Phase 4 of Step 3, the cycle completes. If the process encountered no errors, then the unit will provide an indication of completion of a successful cycle (e.g., the unit may emit a single audible beep). Otherwise, the unit will provide an indication of a failed cycle (e.g., the unit may emit three audible beeps). In either case the indicators (e.g., beep(s)) will repeat until the door is opened or the unit is put into standby by the operator. A maximum process time may be predefined (e.g., the entire process executes within 60 minutes plus the ACT, for example, a maximum of 9 minutes additional).

Communications Interface

In some embodiments, the COM port may be used to program the system and calibrate the system by the manufacturer. The COM port can also be used to download the last Cycle run data that the unit stored. Any standard serial printer or PC with, e.g., HyperTerminal set at 9600/8/N/1 may be used. A PC application may be used to allow the user to connect a PC to the system and download the cycle and service data to the PC and store it in a history file. This program may also be used with passwords to allow more functions as the service log and burn in cycle logs are stored for validation.

Fixed Parameters/Thresholds

| | | |
|---|---|---|
| DOOR INTERLOCK . . . | | |
| Lock: | 50 | Deg-C. |
| Unlock: | 45 | Deg-C. |
| PRE COOL CYCLE . . . | | |
| If Above: | 45 | Deg-C. |
| Cool Down To: | 40 | Deg-C. |
| Cool Down Time (Max): | 22 | Minutes |
| WARM UP CYCLE . . . | | |
| Pre-Warm Up . . . | | |
| Ambient to: | 177 | Deg-C. |
| Pre Warm Time (Max): | 21 | Minutes |
| Warm Up . . . | | |
| Warm Up: | 188 | Deg-C. |
| Warm Up Increment: | 1 | Deg-C./Min. |
| Fail Warm Up (Note 1): | 5 | Deg-C. |
| Warm Time: | 12 | Minutes |
| Post-Warm Up . . . | | |
| Post Warm Up: | 190 | Deg-C. |
| Fail Post Warm Up (Note 1): | 5 | Deg-C. |
| Post-Warm Time (Max): | 2 | Minutes |
| STERILIZATION CYCLE . . . | | |
| sterilize Starts: | 190 | Deg-C. |
| Heat Off: | 190 | Deg-C. |
| Heat On: | 188 | Deg-C. |
| Fail Sterilize (Note 1): | 185 | Deg-C. |
| Sterilization Time: | 3 | Minutes |
| COOL DOWN CYCLE . . . | | |
| Cool Down To: | 40 | Deg-C. |
| Cool Down Time (Max): | 22 | Minutes |
| Additional Cooling (Max): | 9 | Minutes |
| TOTAL CYCLE TIME . . . | | |
| Maximum Time | 60 | Minutes |
| (Excluding ACT) | | |

Note 1:
(Desired - Actual) Temperature

Operator Defined Functions
The user can select from . . .
   Unit: Deg-C and Deg-F
   Door Lock Pin: Enabled or Disabled
   Sounder: Enabled or Disabled
   ACT: Add ional Cooling time
   Print the last cycle log generated
   COM Port: Enabled or Disabled
      Logs

Service Logs may be recorded and stored by the system. An example of such a log is provided.

**** SERVICE LOG ****

| | |
|---|---|
| PCB INFO | |
| Serial No.: | L000078 |
| Tech Name: | GLS |
| Test Date: | Apr. 28, 2007 |
| Action Code: | Rev B |
| SYSTEM INFO | |
| Serial No.: | 1000158 |
| Tech Name: | ABM |
| Test Date: | Jul. 19, 2007 |
| Action Code: | New |
| USER PARAMETERS | |
| Units: | C |
| Door Interlock: | 1 |
| Sound: | 1 |
| Cool Time: | 0 |
| HARDWARE PARAMETERS | |
| Air Filter: | 0 |
| Bagged: | 0 |
| LOWER SET POINT | |
| Calibrated: | Yes |
| Offset: | 0 |
| UPPER SET POINT | |
| Calibrated: | Yes |
| Offset: | −1 |
| DISABLED INTERLOCK | |
| First Cycle: | 0 |
| Last Cycle: | 0 |
| CYCLE DATA | |
| Total Cycles: | 0 |
| COMMENTS: | |

A Cycle Log may also be recorded and stored. An example of such a log is provided:

| | |
|---|---|
| SERIAL NUMBER: | 1000158 |
| CYCLE COUNT: | 1 |
| HEAT | |
| Start Temp: | 26 c. |
| Final Temp: | 190 c. |
| Time: | 21:11 |
| Status: | Pass |
| STERILIZE | |
| Min Temp: | 188 c. |
| Max Temp: | 192 c. |
| Time: | 3:00 |
| Status: | Pass |
| COOL | |
| Start Temp: | 190 c. |
| Final Temp: | 40 c. |
| Time: | 7:58 |
| Status: | Pass |
| COMPLETE | |
| Time: | 32:09 |
| Status: | Pass |
| COMMENTS: | |

Error Codes

The system is programmed to allow ample time to warm up, sterilize, and cool down during a complete cycle. If any stage of this process is interrupted before a cycle has completed, the system will display an error code. The following chart defines example error codes.

Pre-cool down is a phase that only occurs when a cycle is aborted, and the chamber temperature is above 50° C. (135° F.). The system cools down before any other action is permitted to occur, including opening the door if the Safety Door Interlock is set to “on”, the blue LED is lit to indicate cool down.

Error Code Display

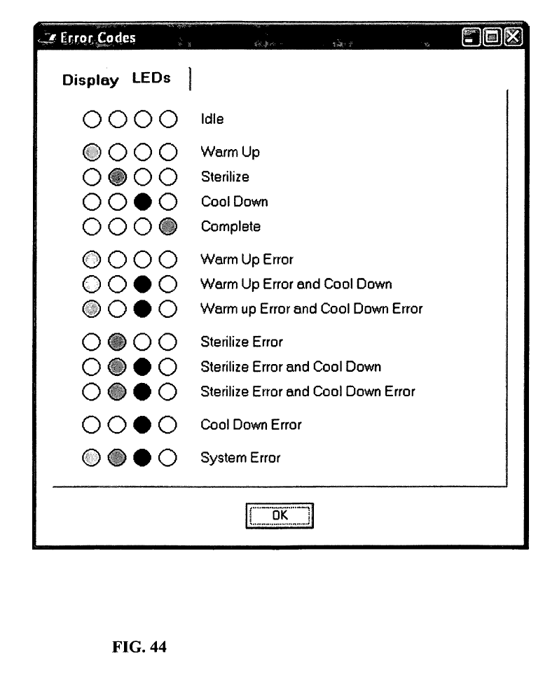

FIG. 44 depicts the LED error codes

INITIAL FAILURES . . .
   E 01—Door Interrupt During Cycle Start
PRE-COOL FAILURES . . .
   E 02—Standby/On Interrupt During Pre Cool
   E 03—Door Interrupt During Pre Cool
   E 04—Cycle Fail During Pre Cool
WARM UP FAILURES . . .
   E 05—Standby/On Interrupt During Warm Up
   E 06—Door Interrupt During Warm Up
   E 07—Cycle Fail During Warm Up
WARM UP FAILURES AND COOL DOWN FAILURES . . .
   E 08—Standby/On Interrupt During Warm Up & Cool Down
   E 09—Door Interrupt During Warm Up & Cool Down
   E 10—Cycle Fail During Warm Up & Cool Down
STERILIZE FAILURES . . .
   E 11—Standby/On Interrupt During Sterilize
   E 12—Door Interrupt During Sterilize
   E 13—Cycle Fail During Sterilize
STERILIZATION FAILURES AND COOL DOWN FAILURES . . .
   E 14—Standby/On Interrupt During Sterilize & Cool Down
   E 15—Door Interrupt During Sterilize & Cool Down
   E 16—Cycle Fail During Sterilize & Cool Down
COOL DOWN FAILURES . . .
   E 17—Standby/On Interrupt During Cool Down
   E 18—Door Interrupt During Cool Down
   E 19—Cycle Fail During Cool Down
SYSTEM ERROR CODES . . .
   E 30—System Error
NONE IDLE FAILURES . . .
   E 31—Supply Power Interrupt During Non-Idle
CONFIGURATION ERROR CODES . . .
   E 40—Low Adjust Failure
   E 41—High Adjust Failure
   E 42—Rtd Failure

E 43—Unit Not Calibrated

| ERROR | | SOLUTION/ACTION |
|---|---|---|
| CODE | ERROR DESCRIPTION | REQUIRED |
| E 01 | Door not closed at cycle start | Close door, press Standby/On switch to |
| | | Standby, then ON, and then press Cycle |
| | | Start |
| E 02 | Standby/On Switch is switched from “On” to “Standby” | Press Standby/On switch to Standby, |
| | during the pre-cool down phase. | then ON, press the Cycle Start switch |
| E 03 | Door is opened during the pre-cool down phase | Close door, press Standby/On switch to |
| | | Standby, then ON, and then press Cycle |
| | | Start |
| E 04 | Cycle parameter fails before pre-cool down phase | Press Standby/On switch to Standby, |
| | (Temperature) | then ON, press the Cycle Start switch |
| E 05 | Standby/On Switch is switched from “On” to “Standby” | Press Standby/On switch to Standby, |
| | during the warm up phase | then ON, press the Cycle Start switch |
| E 06 | Door is opened during the warm up phase | Close door, press Standby/On switch to |
| | | Standby, then ON, and then press Cycle |
| | | Start |
| E 07 | Cycle fails to reach warm up conditions (Temperature) | Press Standby/On switch to Standby, |
| | | then ON, press the Cycle Start switch |
| E 08 | Standby/On Switch is switched from “On” to “Standby” | Press Standby/On switch to Standby, |
| | either in warm up or cool down | then ON, press the Cycle Start switch |
| E 09 | Door is opened during cool down after failure to reach | Close door, press Standby/On switch to |
| | warm up temperature | Standby, then ON, and then press Cycle |
| | | Start |
| E 10 | **Cycle fails to meet warm up and cool down parameters | **Press Standby/On switch to Standby, |
| | (Temperature) | then ON, press the Cycle Start switch |
| E 11 | Standby/On Switch is switched from “On” to “Standby” | Press Standby/On switch to Standby, |
| | during the sterilization phase | then ON, press the Cycle Start switch |
| E 12 | Door is opened during the sterilization phase | Close door, press Standby/On switch to |
| | | Standby, then ON, and then press Cycle |
| | | Start |
| E 13 | Failure to maintain sterilization parameters (Temperature) | Press Standby/On switch to Standby, |
| | | then ON, press the Cycle Start switch |
| E 14 | Standby/On Switch is switched from “On” to “Standby” | Press Standby/On switch to Standby, |
| | during cool down after failure to maintain sterilization | then ON, press the Cycle Start switch |
| | temperature | |
| E 15 | Door is opened during sterilize or cool down | Close door, press Standby/On switch to |
| | | Standby, then ON, and then press Cycle |
| | | Start |
| E 16 | Failure to reach sterilize or cool down parameters | Press Standby/On switch to Standby, |
| | (Temperature) | then ON, press the Cycle Start switch |
| E 17 | Standby/On Switch is switched from “On” to “Standby” | Press Standby/On switch to Standby, then |
| | during cool down | ON, press the Cycle Start switch |
| E 18 | Door is opened during cool down | Close door, press Standby/On switch to |
| | | Standby, then ON, and then press Cycle |
| | | Start |
| E 19 | Failure to reach cool down parameters (Temperature) | Press Standby/On switch to Standby, then |
| | | ON, press the Cycle Start switch |
| E 30 | System error (Catch all) | Contact Manufacturer Service Dept |
| E 31 | Building Power Interrupt | Press Standby/On switch to Standby, then |
| | | ON, press the Cycle Start switch |
| E 40 | Calibration low adjust failure | Contact Manufacturer Service Dept. |
| E 41 | Calibration high adjust failure | Contact Manufacturer Service Dept. |
| E 42 | RTD (Temperature Sensor) failure | Contact Manufacturer Service Dept. |
| E 43 | No calibration | Contact Manufacturer Service Dept. |

If an error occurs, the LED representing the phase of the cycle where the error occurred will be flashing. For example, if the yellow and blue LEDs are flashing, then there was a failure in the warm up phase and in the cool down phase.
Software System Architecture

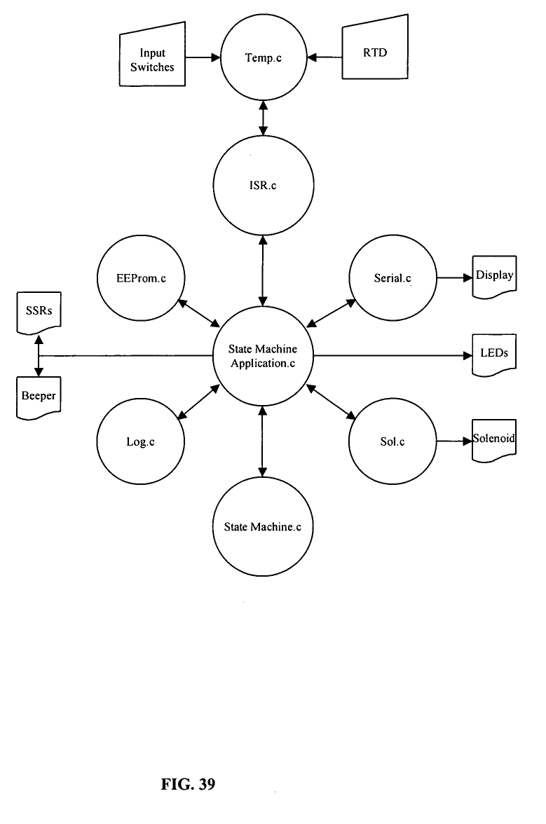

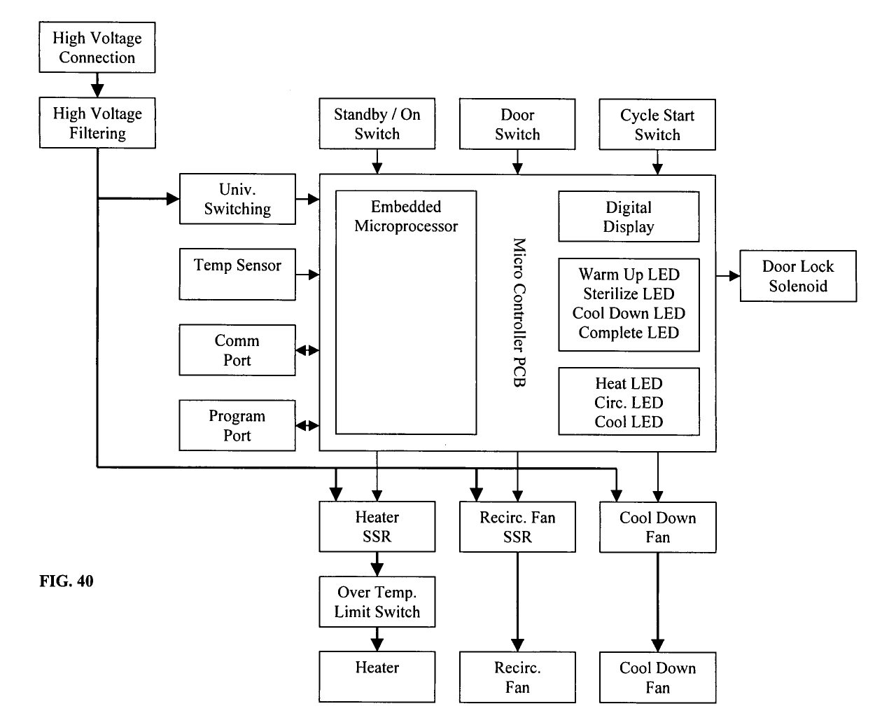

The following modules and their respective functions along with the architecture diagram of FIG. 39 denote and describe an example code structure for the system.

The Eeprom.c module allows the data to be read from or written to the eeprom.

The Isr.c module (Interrupt Service Routine module) provides communication and monitoring of temperature and all switches, drives, displays, LEDs, the door solenoid and the beeper. This is performed at 50 ms intervals.

The Log.c module provides for the cycle log data to be stored so that it is available for printing at the completion of a run.

The Serial.c module controls the serial display driver.

The Solenoid.c module provides functionality of the door solenoid

The StateMachine.c module is the “router”, this section provides the engine but it knows nothing about the modules.

The StateMachineApp.c module provides the behavior, communicates to the outputs, and also defines all the error codes.

The Temperature.c module provides the temperature information for the control signals.

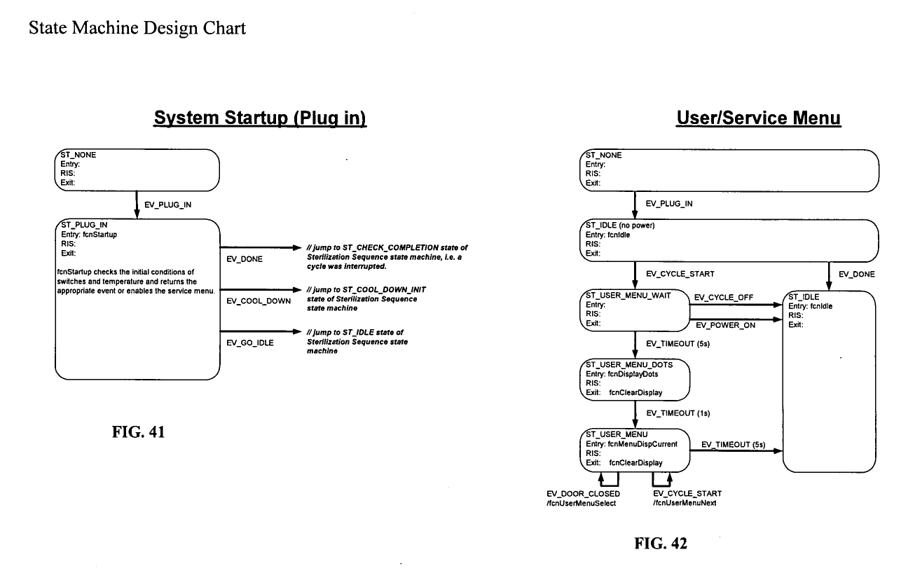

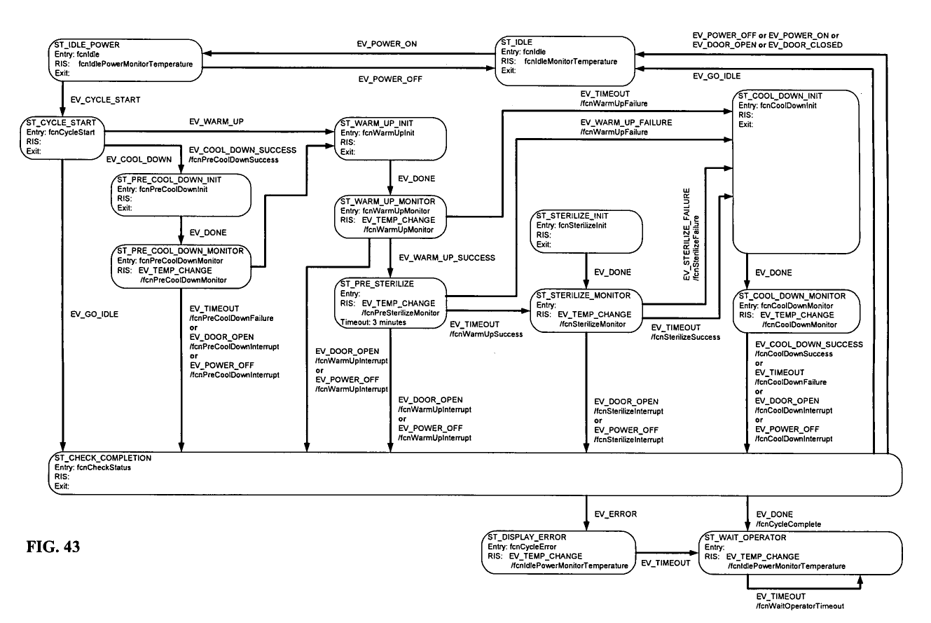

Referring now to the state machines defined in FIGS. 41 to 43, implementation details of the above described processes are further disclosed. A common design specification has been used in developing embodiments of the present invention. In other words, the information contained in this section has been designed to be used in other devices. Specifically, this includes the StateMachine handler. The StateMachine software has been designed to build on the MPLAB platform as well as the Microsoft Visual C++ Version 6.0 platform. Of course, any suitable platform may be used.

Common.h

Contains a few typedefs.

StateMachine Handler

The state machine handler has been designed to allow for a design to be implemented using state behavior. The device is in one and only one state at any instant. The device will move from one state to another by processing an event, which allows it to transition to another state. This design is very explicit and helps to prevent unwanted behavior. By its very nature, any event, which is not handled by the current state, is simply dropped. For most projects, this eliminates the need to specifically exclude or disable actions. A specific example, for system, there is no need to disable the CycleStart button in certain states as long as the EV₁₃CYCLE₁₃START event is not included in the processing for that state. State machine terms are defined in the next sub-sections.

State Machine terms:

States

A particular state that the module can reside in. The module is always in one and only one state. For example, the idle state ST_IDLE.

Events

An event is the stimulus, which may or may not cause the module to transition to a new state. An event can be generated by the hardware interface or the entry, exit or reactions in state code may return an event to be processed. Not all events must be processed, they can be ignored.

Transitions

A transition allows a state machine to move from one state to another and will do so if the event associated with the transition is the current event being processed.
   Reactions in State
A reaction in state is defined as an event, which causes code to be executed without a change in state.
   Code
Code may be associated with a transition, a reaction in state, the entry to a state or the exit of a state but is not required. In other words, table entries may contain a NULL entry. The tables will be explained shortly.

State Machine Tables

Transition Table

| | |
|---|---|
| typedef struct { | |
| WORD CurrentState; | // Current state |
| WORD Event; | // Transition event |
| WORD (*TransitionFunction)(void); | // Transition code |
| WORD NextState; | // Destination state |
| } StateMachineTransitionDefinition; | |

The transition table defines all events (Event) to be processed by a particular state (CurrentState), the code to be run when transitioning (TransitionFunction) and the destination state. The code may be a NULL pointer, which indicates no code is to be run. No attempt has been made to detect duplicate events for a state and the first table entry matching the CurrentState and Event will be processed.

Reactions In State Table

| | |
|---|---|
| typedef struct { | |
| WORD CurrentState; | // Current state |
| WORD Event; | // Reaction in state event |
| WORD (*RISFunction)(void); | // Reaction in state function |
| } StateMachineRISDefinition; | |

The reactions in state table defines all events (Event) to be processed by a particular state (CurrentState) without transitioning to another state. The defined code (RISFunction) will be executed. A NULL pointer may be specified, but since no state transitioning is taking place, the net effect would be zero on the state machine. No attempt has been made to detect duplicate events for a reaction in state and the first table entry matching the CurrentState and Event will be processed. Also, the same CurrentState/Event pair in the reactions in state table will be processed instead of one in the transition table. In other words, the one in the transition table will be ignored.

State Table

| | |
|---|---|
| typedef struct { | |
| WORD State; | // State of state machine |
| WORD (*EntryFunction)(void); | // State entry code |
| WORD (*ExitFunction)(void); | // State exit code |
| WORD StateTimeout; | // State timeout |
| } StateMachineCodeDefinition; | |

The state table defines all entry (EntryFunction) and exit (ExitFunction) code to be processed when entering or exiting a particular state (State). A NULL pointer may be specified for either function and will be simply ignored. The timeout (StateTimeout), if non-zero, will be scheduled when entering the state and will generate a timeout if time expires. The timeout is automatically canceled when leaving the state.
State Flow Rules

The method in which the state machine executes is defined as the state flow or behavior. By envisioning the state flow, which is meant to be very intuitive, the state machine design is created. For this reason, the state machine design is currently limited to one level. I.e. no parent or sub-statecharts. So, the state flow rules are as follows:
1) An event must be generated for any state behavior to execute.
2) If the current state and generated event are matched to the reactions in state table . . .
   a) The reactions in state function is executed.
   b) The processing of the event is complete. That is, no additional processing is executed for this event.
3) Else If the current state and generated event are matched to the transitions table . . .
   a) The current timeout, if any, is canceled.
   b) The transitions table function is stored for later execution.
   c) The transitions table next state is stored.
   d) The state table is searched for the current state and the exit function is stored for later execution.
   e) The state table is searched for the next state and the entry function and next state timeout is stored for later execution.
   f) Now, execution begins . . .
      i) The exit function of the current state is executed if not NULL.
      ii) The transition function is executed if not NULL.
      iii) The physical state is changed to the next state.
      iv) The entry function of the next state is executed if not NULL.
      v) The next state timeout is scheduled if not zero.
4) Else
   a) The event is ignored.
      State Machine Hardware Interface

The state machine hardware interface consists of a timeout handler and module specific hardware polling handler. These are described in more detail in the module specific design section of this document.

StateMachine.c Code Comments

For reference, an example of actual code comments are included here:

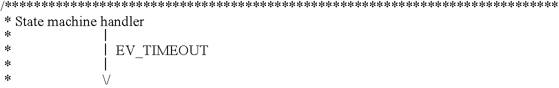

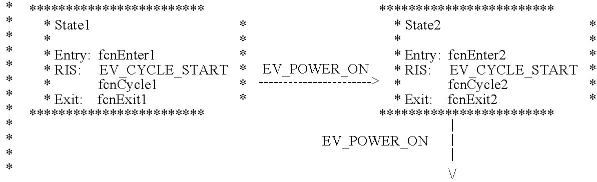

* For the above example:

| | |
|---|---|
| * | 1) The events EV_TIMEOUT and EV_POWER_ON are entries in the SMTransitions table. |
| * | 2) The Entry and Exit functions fcnEnter1, fcnExit1, fcnEntry2 and fcnExit2 are |
| * | entries in the SMCode table. |
| * | 3) The reactions in state EV_CYCLE_START along with functions fcnCycle1 and |
| * | fcnCycle2 are entries in the SMReactionsInState table. |
| * | |

* State flow (not including the states not shown):

| | |
|---|---|
| * | 1) EV_TIMEOUT into State1 will cause State1's Entry code to run --> fcnEnter1 |
| * | 2) In State1, an EV_CYCLE_START event will cause the ReactionInState code to |
| * | run --> fcnCycle1 |
| * | 3) In State1, an EV_POWER_ON event will cause the following code to run, |
| * | State1's Exit --> fcnExit1 and State2's Entry -->fcnEnter2 |
| * | 4) In State2, an EV_CYCLE_START event will cause the ReactionInState code to |
| * | run --> fcnCycle2 |
| * | 5) In State2, an EV_POWER_ON event will cause the following code to run, |
| * | State2's Exit --> fcnExit2 |
| * | 6) . . . AND SO ON! |
| * | |

* State flow rules:

| | |
|---|---|
| * | 1) The same event is not to be defined for both a ReactionInState |
| * | and an Event Transition |
| * | 2) ReactionsInState run first if handled. |
| * | 2a) If handled as RIS, the event is complete. |
| * | 3) Determine if event transition is executed (else it could be ignored) |
| * | |
| * | If transition taken |

| | |
|---|---|
| * | 4) Any previous timeout is canceled |
| * | 5) Exit code of the current state is run. |
| * | 6) Transition code of the event is run. |
| * | 7) State is changed to next state |
| * | 8) Entry code of the next state is run. |
| * | 9) Next state timeout is scheduled |
| * | |

********************************************************************************/

Example Design Specification
Interrupt Service Routines
Timer Channel Interrupt Service Routine
The timer channel interrupt rate is 1 KHz and is vectored to the high priority interrupt.
   UART Interrupt Service Routine
The UART transmit and receive events are vectored to the low priority interrupt.
   State Machine Hardware Interface
   Event Generation and Processing

Event generation for the system is handled by a simple array of 10 Words. There are two indexes into the array, the GetEventIndex used by the main thread to process events and the PutEventIndex used by the interrupt timeout handler and hardware polling handler to generate events. Defines, variables and functions are described below.

```
#define QUEUE_EVENTS_MAX 10
WORD EventQueue[QUEUE_EVENTS_MAX]; // All init to
EV_NONE
BYTE GetEventIndex=0;
BYTE PutEventIndex=0;
void EventGenerate(WORD event)
{
    EventQueue[PutEventIndex] = event;
    PutEventIndex++;
    If(PutEventIndex == QUEUE_EVENTS_MAX)
PutEventIndex = 0;
}
WORD EventRetrieve(void)
{
    WORD return_event = EV_NONE;
    if(EventQueue[GetEventIndex] != EV_NONE)
    {
            // retrieve the event
            return_event = EventQueue[GetEventIndex];
            GetEventIndex++;
            If(GetEventIndex == QUEUE_EVENTS_MAX)
GetEventIndex = 0;
    }
```

Timeout Handler
Timeouts for the system have a granularity of 1 second. The timeout handler has a thread component and an interrupt component.

The thread component can cancel timeouts or set timeouts. Based on access by just one thread and one interrupt, there is no need for interrupt disabling while the timeout is canceled (set to 0) or re-scheduled (set to non-zero).

void smSetTimeout(WORD Seconds)
{
smTimeout = Seconds; // set current
timeout
}

If a timeout is currently scheduled, smTimeout !=0, the interrupt component decrements it and generates a timeout event when the timeout reaches 0. The interrupt component is implemented in the high priority interrupt.

| | |
|---|---|
| // To be called every second | |
| void DrySterlizerTimeoutHandler(void) | |
| { | |
| if(smTimeout != 0) | |
| { | |
| smTimeout−−; | |
| if(smTimeout == 0) | |
| { | |
| smTimeoutEvent = 1; | // |
| Microsoft Visual C++ | |
| EventGenerate(EV_TIMEOUT); | // |
| MPLAB | |
| } | |
| } | |
| } | |

Hardware Polling Handler
The hardware polling handler is implemented the high priority interrupt handler. To prevent conflicts between the main thread and the interrupt hardware polling from trying to manipulate the same variables, the interrupt hardware polling MUST keep its own shadow of the hardware state.
For example, suppose the variable smDoorCondition is set to DOOR_CLOSED and an EV_DOOR_OPEN event is generated (put on the queue) to the main thread. The processing of that event sets a variable smDoorCondition=DOOR_OPEN. However, this event is generated in the 1 ms interrupt. So, if the main thread does not process the original event and update smDoorCondition before the next 1 ms, another event will be generated in the interrupt.

Example of interrupt hardware polling:

```
BYTE smISRDoorCondition = DOOR_CLOSED;
void DrySterilizerHardwarePolling(void)
{
    // Door open is IN_DOOR_SWITCH = CLEAR
    if(IN_DOOR_SWITCH == CLEAR)
    {
        if(smISRDoorCondition != DOOR_OPEN)
        {
            smISRDoorCondition = DOOR_OPEN;
            EventGenerate(EV_DOOR_OPEN);
        }
    }
    else
    {
        if(smISRDoorCondition != DOOR_CLOSED)
        {
            smISRDoorCondition = DOOR_CLOSED;
            EventGenerate(EV_DOOR_CLOSED);
        }
    }
}
```

Main Thread Of Execution
The main thread of execution has several responsibilities.
   Microprocessor Configuration
Microprocessor configuration is executed one time at startup. Please reference the function call InitMicro( ) in the source module DDS7000MainApp.c as this will have the latest information regarding configuration.
   Interrupt Enabling
Interrupt enabling is executed one time at startup.
Please reference the function call Enablelnterrupts( ) in the source module DDS700OMainApp.c as this will have the latest information regarding interrupt enabling.
   Process Events

Events are processed continually in the main thread. The system has no RTOS and cannot pend on a semaphore or message queue. The mechanism for retrieving an event is the function EventRetrieve to return an event different from EV_NONE. The main processing loop is as follows:

```
WORD next_event = EV_NONE;
while(1)
{
  if(next_event != EV_NONE)
  {
    // event returned from previous event
processing takes
    // precedence over new messages on queue.
    // event processing could continually return
events
    // but is not expected to return more than 1
event
    next_event =
  StateMachineHandler(next_event);
  }
  else if(EventRetrieve( ) != EV_NONE)
  {
    // once any previous message processing has
run
    // to steady state, the queue is polled.
    next_event =
  StateMachineHandler(next_event);
  }
}
```

Hardware Control

System hardware control is handled through the fcnControl function. It is simply a large switch statement on each hardware control element define. The control element and desired state is passed in. If the desired state does not match the current state, the control element is actuated to the desired state. A global variable is used to indicate the current state of the control element. This global is also available anywhere in the system when the control element state is needed. For example, in the fcnPlugIn function, it will use the current state of the door, cycle start switch and on/off switch.

| | |
|---|---|
| // .. this group must be unique | |
| #define CONTROL_DOOR | 1 |
| #define CONTROL_HEATER | 2 |
| #define CONTROL_INTERNAL_FAN 3 | |
| void fcnControl(BYTE control_element, BYTE | |
| newstate) | |
| { | |
| switch(control_element) | |
| { | |
| case CONTROL_DOOR: | |
| if(newstate == DOOR_UNLOCKED) | |
| { | |
| if(smDoorCondition == DOOR_LOCKED) | |
| { | |
| // unlock it | |
| // update state | |
| smDoorCondition = | |
| DOOR_UNLOCKED; | |
| } | |
| } | |
| else if(newstate == DOOR_LOCKED) | |
| { | |
| if(smDoorCondition == | |
| DOOR_UNLOCKED) | |
| { | |
| // lock it | |
| // update state | |
| smDoorCondition = | |
| DOOR_LOCKED; | |
| } | |
| } | |
| break; | |

State Machines
Controllers
Beeper Thread Control
Beeper control in general is configured (enabled or disabled) through the user menu. All attempts to actuate the beeper will be ignored if the beeper configuration is disabled.
The system may have just 1 beeper. The successful completion of a cycle is indicated by a one second beep. This special one second beep is handled by the actual state machine. The completion of a cycle with an associated error condition is indicated by three half-second beeps at one second intervals.
Any number of beeps may be programmed by calling the fcnBeeperSetup function with the desired number of beeps. This function does not actually turn on the beeper but sets up the beep count.
   Beeper Interrupt Control
The actual enabling of the beeper is performed by the function fcnBeeperDriver. This function is called every millisecond by the high level interrupt. The beeper is turned on when the current millisecond count is 0 and turned off when the count is 500.
   Display Thread Control
The system display includes a 4-segment LED display. The firmware sends the display characters via SPI bus to a CPLD which then drives the physical display.

The design allows for 5 bits to define each character which yields a total of 32 characters. The character set showing decimal value, hexadecimal value and character is as follows. Note, some characters may be displayed in Upper Case and some are Lower Case:

```
  0: 00: 0
  1: 01: 1
  2: 02: 2
  3: 03: 3
  4: 04: 4
  5: 05: 5
  6: 06: 6
  7: 07: 7
  8: 08: 8
  9: 09: 9
10: 0A: A
11: 0B: b
12: 0C: c
13: 0D: d
14: 0E: E
15: 0F: F
16: 10: G
17: 11: H
18: 12: . (blank)
19: 13: J
20: 14: —(dash)
21: 15: L
22: 16: _(under)
23: 17: n
24: 18: o
25: 19: P
26: 1A: [ (bracket)
27: 1B: r
28: 1C: ] (bracket)
29: 1D: t
30: 1E: u
31: 1F: y
```

Example, the 32-bit hexadecimal value to display the word “HELP” is 0x10E1519
   The system will support three display “slots”, 0 through 2. These “slots” will be displayed to the actual display device in a round robin fashion. If a slot is empty, the item from the last non-empty slot will be used. No attempt to provide equal time will be made. Worst case, if two of the three display slots are used, the display time will be at a 2:1 ratio.
The function WriteSegmentDisplay takes slot and data. Generally, for the system, the display slots are used for the following purposes:
Slot 0—Menus and temperature during cycles
Slot 1—Burn in information
Slot 2—Error and completion information
   Display Interrupt Control

The function SegmentDisplayDriver is called once per second from the ISR, with the current Seconds count, to update the display. If follows the following rules:
1) Use the timestamp (in seconds) passed in mod the number of display slots to determine the next slot to display.
2) If the slot has data, display it.
3) If the slot is empty and all slots are empty, clear the display
4) If the slot is empty and all slots are NOT empty, display the data for the last slot with data. This will give slot 2 more precedence whenever it has data.
   So, from these rules, here are a few possible scenarios:
1) All slots are filled. Each will get equal time on the display.
2) Only one slot has data and will get all the time on the display.
3) Slots 1 and 3 have data, slot 3 gets two-thirds of the display time.

When SegmentDisplayDriver has determined the actual data to be sent to the display, the function SendToDisplayDriver is called. It's function is to build the 45-bit characters into a 20-bit stream and send to the SPI port.

Door Solenoid Thread Controller

The door solenoid control is configured (enabled or disabled) through the user menu which calls the function fcnSolenoidEnable. All attempts to actuate the solenoid will be ignored if the solenoid configuration is disabled. Next, solenoid control is activated during a cycle by calling the function fcnSolenoidactivate. Therefore, the solenoid must be enabled and activated before the solenoid interrupt controller could actually lock the door.

Two additional variables to determine solenoid control are the current temperature and the current state of the door switch. Using configuration, activation, temperature and door switch yields a total of 16 cases. The collapsed truth table is as follows:

| | | Current | Door | |
|---|---|---|---|---|
| Enabled | Activate | Temp | Switch | Action |
| No | n/a | n/a | n/a | Door Unlocked (8 cases) |
| Yes | No | n/a | n/a | Door Unlocked (4 cases) |
| Yes | Yes | >Lock Temp | Open | Door Unlocked (1 case) |
| | | | | Cannot lock door if open |
| Yes | Yes | >Lock Temp | Closed | Door Locked (1 case) |
| Yes | Yes | <Unlock Temp | n/a | Door Unlocked (2 cases) |
| | | | | Total: 16 cases |

Door Solenoid Interrupt Controller

The function fcnSolenoidMonitor is the main solenoid control function and is called once per second from the high level interrupt. Keep in mind this function will not necessarily exit immediately if the solenoid is disabled or deactivated. The disabling or deactivation may have occurred while the door was locked and it will now be required that the door be unlocked. Therefore, all calls to the solenoid monitoring function will check the current state of the door lock versus the above truth table to determine if any action is necessary.

Once an action is required on the solenoid, the solenoid relay is actuated for the proper direction of the solenoid and the solenoid itself is actuated. A timeout is setup for the solenoid de-actuation which currently occurs after 1 s. At this point another timeout is setup for the relay de-actuation which is also occurs at 1 s. When the relay time expires, the entire solenoid action is completed.

Another point to be made about the solenoid monitor function is that no additional checking for solenoid actions will be made until any current actions have completed.

LED Thread Control

The system may be implemented with just 4 LEDs used for consumer indicators. These are the WarmUp, Sterilize, CoolDown and CycleComplete LEDs. A flashing LED indicates an improperly completed portion of the cycle. In other words, if the warm up fails but the cool down is successful, the WarmUp LED will be flashing and the CoolDown LED will remain on steady at the end of the cycle. A successful sterilization cycle (complete) will result in the CycleComplete LED on steady at the completion of the cycle. The completion of a cycle, whether successful or not, will leave the resultant LEDs on until the door is opened or the power is switched off. The combination of 4 LEDs and the requirement to flash has allowed for the entire LED control to be encoded into just one byte. Calling the fcnControl function described earlier performs LED control. It enables or disables LED bits, but does not actually turn them on or flash them.

LED Control Byte

| Flashing 1 Hz | | | | On Steady | | | |
|---|---|---|---|---|---|---|---|
| B7 | B6 | B5 | B4 | B3 | B2 | B1 | B0 |
| Failed to | Failed to | Failed to | N/A | Cool | Sterilization | Warm | Cycle |
| reach cool | maintain | reach | | Down In | In Progress | Up In | Complete |
| down | temperature | sterilization | | Progress | | Progress | |
| temperature | during | temperature | | | | | |
| | sterilization | | | | | | |

Example code:

| | |
|---|---|
| #define LED_WARM_UP | 0x02 |
| // On | |
| #define LED_ERROR_MIN_TEMP_NOT_REACHED | 0x20 |
| // Flashing | |
| // normal warm up in progress | |
| fcnControl(CONTROL_LED_ON, LED_WARM_UP); | |
| // warm up failed, change to flashing LED | |
| fcnControl(CONTROL_LED_OFF, LED_WARM_UP); | |
| fcnControl(CONTROL_LED_ON, | |
| LED_ERROR_MIN_TEMP_NOT_REACHED); | |

LED Interrupt Control
The LEDs are physically controlled by the function fcnLEDDriver, which will turn on, turn off or flash (1 flash per second) the LEDs. This function is called every millisecond by the high level interrupt.
fcnLEDDriver takes one parameter, the current MilliSecond count. The flashing bit takes precedance over the on bit. Therefore, an LED with both the flashing and on bits set will flash. A flashing LED will be on when the current MilliSecond count is <=500 and off when greater than 500.
   Temperature Control
The function fcnCalculateTemperature converts the actual A2D reading to a linear and scaled value that represents the actual temperature in degree-C.
fcnCalculateTemperature—calculates based on RTD reading, voltage lookup
Steps to calculating actual temperature (example A2D reading of 765):
1) Lookup the current A2D reading and retrieves the nearest values
   160c=>745(a2d) and 170c=>779(a2d)
2) Perform an interpolation from those values to calculate actual temperature

$$temp = [ LoTemp + \frac{( ActualA 2 D - LoA 2 D ) * 10}{HiA 2 D - LoA 2 D} ]$$

$$temp = [ 160 + \frac{( 765 - 745 ) * 10}{779 - 745} ]$$

$$temp = 165.882$$
3) Perform an offset adjustment—see fcnAdjustTemperature function

Lookup Table:

const TemperatureVoltageLookup tvl[TVL_LOOKUP_COUNT] =
{

| | | | |
|---|---|---|---|
| 0, | 0, | 90, | 472, |
| 10, | 65, | 100, | 515, |
| 20, | 123, | 110, | 557, |
| 30, | 179, | 120, | 597, |
| 40, | 232, | 130, | 636, |
| 50, | 284, | 140, | 673, |
| 60, | 334, | 150, | 709, |
| 70, | 382, | 160, | 745, |
| 80, | 428, | 170, | 779, |
| 180, | 812, | | |
| 190, | 844, | | |
| 200, | 875, | | |
| 210, | 905, | | |
| 220, | 934, | | |
| 230, | 963, | | |
| 240, | 990, | | |
| 250, | 1017, | | |

};

The function fcnAdjustTemperature compensates for the hardware stack up tolerance errors. fcnAdjustTemperature adjusts temperature based on zero and hi-limit adjustments.
1) Adjustments made to all temperatures will be based on a straight-line Extrapolation of the hi and lo limit adjustment values.
2) LO_LIMIT_TEMPERATURE=20 and HI_LIMIT_TEMPERATURE=220 for the below documentation
3) The multiplies and divides by 1000 is used for integer math.
   Steps to Adjusting the Temperature:

Note: IMM: Integer math multiplier -->100,000
1) Determine the 0 degrees offset temperature, i.e. draw a straight line through the offsets and get the y-offset. This is accomplished by determining the percentage of the offset of lo vs hi and subtracting from lo.

$$Percent 1000 = \frac{smLoLimitTemp * IMM}{smHiLimitTemp - smLoLimitTemp}$$

$$ZeroLimitAdjust = \frac{( smLoLimitAdjust - smHiLimitAdjust ) * Percent 1000}{IMM} + smLoLimitAdjust$$
2) Calculate the per degree offset

$$PerDegreeOffset 1000 = \frac{( smLoLimitAdjust - smHiLimitAdjust ) * IMM}{smHiLimitTemp - smLoLimitTemp}$$
3) Calculate final temperature. Final temperature equals interpolated temperature from the lookup table normalized to 0 (add ZeroLimitAdjust) plus offset per degree times PerDegreeOffset1000.
   Switch Control
   Input switches shall be serviced within 100 mS and must have a denounce time less then 100 mS.
      Logging

Logging Sample

**** SERVICE LOG ****
FIRMWARE VERSION: 01.04

| | |
|---|---|
| PCB INFO | |
| Serial No.: | L00004 |
| Tech Name: | LIB |
| Test Date: | Feb. 14, 2007 |
| Action Code: | Rev.B |
| SYSTEM INFO | |
| Serial No.: | 100007 |
| Tech Name: | GLS |
| Test Date: | Feb. 23, 2007 |
| Action Code: | New |
| USER PARAMETERS | |
| Units: | C |
| Door Interlock: | 1 |
| Sound: | 1 |
| Cool Time: | 0 |
| HARDWARE PARAMETERS | |
| Air Filter: | 0 |
| Bagged: | 0 |
| LOWER SET POINT | |
| Calibrated: | Yes |
| Offset: | 0 |
| UPPER SET POINT | |
| Calibrated: | Yes |
| Offset: | −1 |
| DISABLED INTERLOCK | |
| First Cycle: | 0 |
| Last Cycle: | 0 |
| CYCLE DATA | |
| Total Cycles: | 17 |
| COMMENTS: | |
| SERIAL NUMBER: | 100007 |
| CYCLE COUNT: | 17 |
| HEAT | |
| Start Temp: | 46 c. |
| Final Temp: | 190 c. |
| Time: | 17:53 |
| Status: | Pass |
| STERILIZE | |
| Min Temp: | 188 c. |
| Max Temp: | 191 c. |
| Time: | 3:00 |
| Status: | Pass |
| COOL | |
| Start Temp: | 188 c. |
| Final Temp: | 40 c. |
| Time: | 7:58 |
| Status: | Pass |
| COMPLETE | |
| Time: | 28:51 |
| Status: | Pass |
| COMMENTS: | |

Log Recording

The cycle log will be recorded to EEPROM in four-byte records. The theoretical maximum amount of log space will be cleared to 0's at the beginning of a cycle. Therefore, a log record of type 0 will be considered invalid and also the end of the current log.

Log Record

| B31-B24 | B23-B0 |
|---|---|
| Log Record | Log Record Data |
| Type | |

The types of log records are shown in the following table along with the numerical value for each and the location (in software) where the information will be recorded.

| Log Record Type | Val | What | Where |
|---|---|---|---|
| LOG_END | 0 | Invalid, therefore end | Memory cleared |
| | | of log | |
| LOG_SERIAL_NUMBER | 1 | Device serial number | Warm init |
| LOG_CYCLE_COUNT | 2 | Cycle count high | Warm init |
| LOG_CYCLE_TYPE | 3 | Which portion of cycle | Warm/Ster/Cool init |
| LOG_TEMP_START | 4 | Temp at start of cycle | Portion init |
| | | portion | |
| LOG_TEMP_FINAL | 5 | Temp at cycle portion | Portion success/fail |
| | | end | |
| LOG_TEMP_MIN | 6 | Minimum portion | Portion init/monitor |
| | | temp | |
| LOG_TEMP_MAX | 7 | Maximum portion | Portion init/monitor |
| | | temp | |
| LOG_PORTION_TIME | 8 | Time of current | Portion success/fail |
| | | portion | |
| LOG_PORTION_STATUS | 9 | Pass/fail for portion of | Portion success/fail |
| | | cycle | |
| LOG_ERROR_CODE | 10 | Error code for the | Cycle completion error |
| | | cycle | code |

The number of expected log entries for each type are as follows

| Log Record Type | Count | Notes |
|---|---|---|
| LOG_END | x | Remainder of log |
| LOG_SERIAL_NUMBER | 1 | 1 S/N |
| LOG_CYCLE_COUNT | 1 | 1 entry |
| LOsG_CYCLE_TYPE | 4 | 3 portions |
| LOG_TEMP | 8 | 2 (pre-cool) + 2 |
| | | (warm) + 2 |
| | | (sterilize) + 2 |
| | | (cool) |
| LOG_PORTION_TIME | 5 | 4 portions + complete |
| LOG_PORTION_STATUS | 5 | 4 portions + compplete |
| TOTAL | 24 | *4 = 96 total log bytes |

Log Behavior
The log behavior is defined as follows:
   The log is cleared at the beginning of every cycle. This automatically leaves the last cycle's log intact until another cycle is started, thereby allowing it to be retrieved as many times as needed.
   Each log entry is immediately written to the EEPROM. This allows a partial log to be retrieved if the device is power off during the cycle.
   The log is automatically dumped to the serial port upon cycle completion.
   Logging APIs are never to be called from interrupt context. Protection is automatic with the state machine engine. I.e. there is only 1 thread in the state machine.
   The log output will not necessarily contain information for all parts of the cycle if they did not execute due to an error condition.
      PC vs Hardware Control
The module software has been designed to allow the device to be controlled from a connected PC keyboard or the actual module hardware. This is in reference to the control inputs only. That is, the pc can simulate plugging the device in, powering it on or off, opening or closing the door, hitting the cycle start button and changing the temperature read by the module. This allows complete cycles and test scenarios to be executed from the PC keyboard. In some embodiments, a program running on the PC can execute complete control over the system.
Baud rate and handshaking parameters shall be set to:
   9600 baud/8 data bits/No Parity/1 Stop Bit
      PC Control Enabled
PC control, the default, is enabled by sending a ‘P’ to the device. The serial cable must be plugged in for the device to receive this character.
   Hardware Control Enabled

Hardware control is enabled by simply sending an, ‘H’ to the device. The serial cable must be plugged in.

Control Table

| Control Element | PC Control | HW Control |
|---|---|---|
| Plug Out | ‘0’ | ‘0’ (Actual power plug at |
| | | release) |
| Plug In | ‘1’ | ‘1’ (Actual power plug at |
| | | release) |
| | ‘2’ | |
| | ‘3’ | |
| Power On | ‘4’ | Power Switch to On |
| Door Closed | ‘5’ | Door Closed |
| Cycle Start | ‘6’ | Cycle Start Pressed |
| Power Off | ‘7’ | Power Switch to Off |
| Door Open | ‘8’ | Door Opened |
| Cycle Start - | ‘9’ | Cycle Start Released |
| Released | | |
| Timeout | ‘.’ | Time expires |
| Temperature − 10 | ‘/’ | N/A |
| Temperature + 10 | ‘*’ | N/A |
| Temperature − 1 | ‘−’ | Actual RTD − 1 degree C. |
| Temperature + 1 | ‘+’ | Actual RTD + 1 degree C. |
| Temperature | <CR> - Temp to | N/A |
| Ambient/ | ambient or temp to | |
| Temperature | sterilize temp if | |
| Sterilize | sterilizing. | |
| Select PC Control | N/A | ‘P’ |
| Select HW Control | ‘H’ | N/A |

Test Cases Using PC Control

Normal Cycle

| | | | LEDs | | | | |
|---|---|---|---|---|---|---|---|
| Key | Action | Display | W | S | C | C | Notes |
| ‘P’ | PC Control | empty | | | | | |
| ‘1’ | Plug in | empty | 0 | 0 | 0 | 0 | |
| ‘4’ | Power on | empty | | | | | |
| ‘5’ | Door closed | empty | | | | | |
| <CR> | Set ambient temp | empty | | | | | |
| ‘6’ | Start the sterilization cycle | 22 c | 1 | | | | |
| <CR> | Jump to sterilization temp | 197 c | | 1 | | | |
| ‘.’ | Timeout, sterilization | 197 c | | | 1 | | |
| | successful | | | | | | |
| <CR> | Jump to ambient, cool down | 22 c | | | | 1 | 1 second beep |
| | success | | | | | | |
| ‘8’ | Open door, remove utensils | empty | 0 | 0 | 0 | 0 | |
| ‘5’ | Close door | empty | | | | | ‘6’ to start |
| | | | | | | | another cycle |

Warm Up Fail Cycle

| | | | | | | | |
|---|---|---|---|---|---|---|---|
| ‘P’ | PC Control | empty | | | | | |
| ‘1’ | Plug in | empty | 0 | 0 | 0 | 0 | |
| ‘4’ | Power on | empty | | | | | |
| ‘5’ | Door closed | empty | | | | | |
| <CR> | Set ambient temp | empty | | | | | |
| ‘6’ | Start the sterilization cycle | 36c | 1 | | | | |
| ‘*’ | Temp + 10 | 46c | 1 | | | | |
| ‘*’ | Temp + 10 | 56c | 1 | | | | |
| ‘.’ | Timeout, warm up fail, start | 56c | F | | 1 | | |
| | cool down | | | | | | |
| <CR> | Jump to ambient, cool down | 36c | | | | 1 | 3.5 second beeps |
| | success | | | | | | |
| ‘8’ | Open door, remove utensils | empty | 0 | 0 | 0 | 0 | |
| ‘5’ | Close door | empty | | | | | ‘6’ to start another |
| | | | | | | | cycle |

Auto Burn-in Functionality
Additional functionality is added to allow the unit to perform a number of “burn-in” cycles during production. The details for burn-ins are as follows:
   A maximum count of 9 burn-ins can be programmed in using the service menu and the PC utility.
   When using the PC utility, the character “L” (for Life Test) in the “Cycles” text box of the “Burn-in” tab will translate to a cycle count of 255 (0xff) which the device will interpret as FOREVER.
   Immediately upon exiting the service menu, closing the door, powering up the unit and hitting cycle start, the burn-ins will begin. To minimize coding for burn-ins, if the door is not closed when selecting cycle start, the normal error code for door open will be shown and all burn-ins canceled.
   Each burn in cycle will be 60 minutes in length. The cycle time will be padded from the actual cycle completion time up to the full 60 minutes.
   For each cycle which completes, the burn in count will be decremented and the number of burn in cycles will be incremented in the EEPROM.
   A cycle error will terminate the burn-in process and follow normal cycle error processing.
   During burn-ins, the display will show “run” and the current burn in count. i.e. “run9” indicates that there are a total of 9 cycles, including the current one running, to be completed.
   Successful completion of all burn-ins will display a ‘PASS’
      Additional Features
      Quick Test on the Service Menu
The Quick Test feature, when selected from the service menu, all control elements are exercised in the following order.
   All segment displays will display “8”.
   The door is locked and unlocked.
   The heater and internal fan are enabed for 10 seconds, then disabled.
   The cool down and internal fans are enabled for 10 seconds, then disabled.
   The beeper is enabled for 1 second.
   The display is cleared.
      Cool/Fans on the Service Menu

This feature was added to aid in the temperature and certification testing. When selected, the internal and cool down fans will remain on until de-selected. No other options or controls are active at this point.

Additional Tool Chill Time feature

This feature allows the end user so specify additional cooling time at the end of a sterilization cycle. This time can be from 0 (default) to 9 minutes and can be changed in the service menu.

User/Service Menu

The User/Service menu is entered by holding the CycleStart button for 3 seconds when the module is powered off. There are User selections and Service selections. The User selections are always present while the Service selections are only present if the CycleStart button was held with the Power button off and the Door open when the unit was plugged into the wall outlet.

Once the menu is entered the CycleStart button will traverse the User/Service selections. In this mode the power switch does actually power on or off the unit. Closing the door switch or powering on the unit will ‘select’ the current menu item. Opening the door or powering off the unit will have no effect.

The Menu Selections are as Follows:

User Menu

| Item | Selection | Displays |
|---|---|---|
| Temperature | Toggles temperature units | dEGC or dEGF |
| Units | between Celcius and | |
| | Fahrenheit | |
| Door Lock | Toggles door lock between | Loc1 or Loc0 |
| | lock enabled and disabled | |
| Beeper | Toggles beeper between | Snd1 or Snd0 |
| Enable | enabled and disabled | |
| Tool Chill | Selects additional minutes | CHL0 before selection |
| | of cool down to allow the | CHL1 through CHL9 when |
| | tools to chill further | selected one to many times. |
| | | Wraps to 0 |
| Print Log | Prints the last log | PLOG |
| Commu- | Toggles between no comm, | cOFF or cPrt or cPC |
| nication | printer attached or pc | |
| | attached | |

Service Menu (if service menu enabled)

| Item | Selection | Displays |
|---|---|---|
| Set Low | Sets the Low calibration | LSEt before selection |
| Calibration | value (resistance for 20 C) | Lttt where ttt = temperature |
| | | once the calibration has been |
| | | selected |
| Set High | Set the High calibration | HSEt before selection |
| Calibration | value (resistance for 220 C) | Httt where ttt = temperature |
| | | once the calibration has been |
| | | selected |
| Quick Test | Runs a quick test of all | tESt before selected |
| | LEDs, fans, heater and | 8888 when selected |
| | beeper | |
| Cool Down | Turns on all fans until | cool before selected |
| | selected again - no other | FanS when selected |
| | operations are available | |
| | until selected again | |
| Burn-in | Increments the number of | run0 before selection |
| Cycles | burn-in cycles to run | run1 through run9 when |
| | | selected one to many times. |
| | | Wraps to 0 |

PC Test Application Information

The following information may be used in communication with the system.

| Information | | | Saved to | |
|---|---|---|---|---|
| Area | Item | Direction | EEPROM w/size | Structure |
| Display | Temperature | To pc during cycle | no | CycleInfo |
| Display | Cycle Time | To pc during cycle | no | CycleInfo |
| Switches | Power On | To pc during cycle | no | CycleInfo |
| Switches | Air Filter | To pc during cycle | no | CycleInfo |
| Switches | Door Closed | To pc during cycle | no | CycleInfo |
| Switches | Cycle Start | To pc during cycle | no | CycleInfo |
| Solenoid | Enabled | To pc during cycle | no | CycleInfo |
| Solenoid | Direction | To pc during cycle | no | CycleInfo |
| Solenoid | Engaged | To pc during cycle | no | CycleInfo |
| Solenoid | Feedback | To pc during cycle | no | CycleInfo |
| Cycle | Pre Cool | To pc during cycle | no | CycleInfo |
| | Heat | | | |
| | Sterilize | | | |
| | Cool Down | | | |
| | Complete | | | |
| Device | Heater On | To pc during cycle | no | CycleInfo |
| Device | Internal Fan | To pc during cycle | no | CycleInfo |
| Device | Cooling Fan | To pc during cycle | no | CycleInfo |
| Menu | Control | Both | no | SimpleCommand |
| Menu | All discrete pc | To device | no | SimpleCommand |
| | controls | | | |
| Configuration | Units | Both | yes/1 | Get/SetConfiguration |
| Configuration | Serial Number | Both | yes/7 | Get/SetConfiguration |
| Configuration | Test Date | Both | yes/6 | Get/SetConfiguration |
| Configuration | Technician Name | Both | yes/3 | Get/SetConfiguration |
| Configuration | Action Code | Both | yes/6 | Get/SetConfiguration |
| Configuration | Safety Interlock | Both | yes/1 | Get/SetConfiguration |
| Calibration | Lower Set Point | To device | yes/3 | Calibrate |
| Calibration | Upper Set Point | To device | yes/3 | Calibrate |
| Parameters | TBD | | no | |
| Matrix | Pre Cool Time | To pc | no | LogInfo |
| Matrix | Warm Up Time | To pc | no | LogInfo |
| Matrix | Sterilize Time | To pc | no | LogInfo |
| Matrix | Cool Down Time | To pc | no | LogInfo |
| Matrix | Complete Time | To pc | no | LogInfo |
| Matrix | Cycle Count | Both | yes/3 | Get/SetMetrics |
| Matrix | Interlock Disable First | Both | yes/3 | Get/SetMetrics |
| Matrix | Interlock Disable Last | Both | yes/3 | Get/SetMetrics |

Error Codes

The following is a listing of example configuration, system and cycle completion error codes. When an error code is, initiated, the display may toggle between the actual chamber temperature and the error code.

| | |
|---|---|
| EC_NONE_FOUND | 0 |
| // Initial failures | |
| EC_DOOR_INTERRUPT_CYCLE_START | 1 |
| // Pre-cool failures | |
| EC_POWER_INTERRUPT_PRE_COOL | 2 |
| EC_DOOR_INTERRUPT_PRE_COOL | 3 |
| EC_CYCLE_FAIL_PRE_COOL | 4 |
| // Warm up failures | |
| EC_POWER_INTERRUPT_WARM_UP | 5 |
| EC_DOOR_INTERRUPT_WARM_UP | 6 |
| EC_CYCLE_FAIL_WARM_UP | 7 |
| // Warm up failures and cool down failures | |
| EC_POWER_INTERRUPT_WARM_UP_COOL_DOWN | 8 |
| EC_DOOR_INTERRUPT_WARM_UP_COOL_DOWN | 9 |
| EC_CYCLE_FAIL_WARM_UP_COOL_DOWN | 10 |
| // Sterilize failures | |
| EC_POWER_INTERRUPT_STERILIZE | 11 |
| EC_DOOR_INTERRUPT_STERILIZE | 12 |
| EC_CYCLE_FAIL_STERILIZE | 13 |
| // Sterilization failures and cool down failures | |
| EC_POWER_INTERRUPT_STERILIZE_COOL_DOWN | 14 |
| EC_DOOR_INTERRUPT_STERILIZE_COOL_DOWN | 15 |
| EC_CYCLE_FAIL_STERILIZE_COOL_DOWN | 16 |
| // Cool down failures | |
| EC_POWER_INTERRUPT_COOL_DOWN | 17 |
| EC_DOOR_INTERRUPT_COOL_DOWN | 18 |
| EC_CYCLE_FAIL_COOL_DOWN | 19 |
| // System Error Codes | |
| EC_SYSTEM_ERROR | 30 |
| EC_BUILDING_POWER_INTERRUPT | 31 |
| // Configuration Error Codes | |
| EC_LOW_ADJUST_FAIL | 40 |
| EC_HIGH_ADJUST_FAIL | 41 |
| EC_RTD_FAIL | 42 |
| EC_UNIT_NOT_CALIBRATED | 43 |

While the present invention has been described at some length and with some particularity with respect to the several described embodiments, it is not intended that it should be limited to any such particulars or embodiments or any particular embodiment, but it is to be construed with references to the appended claim so as to provide the broadest possible interpretation of such claim in view of the prior art and, therefore, to effectively encompass the intended scope of the invention. Furthermore, the foregoing describes the invention in terms of embodiments foreseen by the inventor for which an enabling description was available, notwithstanding that insubstantial modifications of the invention, not presently foreseen, may nonetheless represent equivalents thereto.

The invention claimed is:

1. A system comprising:
an enclosure;
a sterilizing chamber defined within the enclosure;
a controller housed within the enclosure and adapted to control a temperature within the sterilizing chamber; and
a memory coupled to the controller and adapted to store executable instructions to direct the controller to:
heat the sterilizing chamber according to a predefined heating profile,
sterilize a load held in the sterilizing chamber for a predefined time period at a predefined temperature, and
cool the sterilizing chamber according to a predefined cooling profile using filtered cool air,
wherein the system further includes a temperature sensor coupled to the controller and disposed to be able to sense the temperature within the sterilizing chamber, and
wherein the controller is further adapted to monitor an output of the temperature sensor and maintain the temperature within the sterilizing chamber according to the predefined heating profile, the predefined temperature, or the predefined cooling profile, and
wherein the controller is further adapted to specify an error condition if the controller fails to maintain the temperature within the sterilizing chamber according to the predefined heating profile, the predefined temperature, or the predefined cooling profile.

2. The system of claim 1, wherein the predefined heating profile includes a first heating rate for a first heating period and a second heating rate for a second heating period.

3. The system of claim 2, wherein the first heating rate is higher than the second heating rate.

4. The system of claim 2, wherein the predefined heating profile includes a stepwise incremental heating profile during the second heating period.

5. The system of claim 4, wherein the stepwise incremental heating profile includes alternately increasing the temperature within the sterilizing chamber by a predefined increment and holding the temperature within the sterilizing chamber for a predefined duration.

6. The system of claim 5, wherein the predefined increment is about one degree Celsius and the predefined duration is about one minute.

7. The system of claim 1, wherein the predefined time period is about three minutes and the predefined temperature is about 190 degrees Celsius.

8. A heat sterilization method, comprising the steps of:
heating a sterilizing chamber according to a predefined heating profile;
sterilizing a load held in the sterilizing chamber for a predefined time period at a predefined temperature; and
cooling the sterilizing chamber according to a predefined cooling profile using filtered cool air, wherein
the predefined heating profile includes a first heating rate for a first heating period and a second heating rate for a second heating period, and
the first heating rate is higher than the second heating rate.

9. The method of claim 8, wherein the predefined heating profile includes a stepwise incremental heating profile during the second heating period.

10. The method of claim 9, wherein the stepwise incremental heating profile includes alternately increasing the temperature within the sterilizing chamber by a predefined increment and holding the temperature within the sterilizing chamber for a predefined duration.

11. The method of claim 10, wherein the predefined increment is about one degree Celsius and the predefined duration is about one minute.

12. The method of claim 8, wherein the predefined time period is about three minutes and the predefined temperature is about 190 degrees Celsius.
